(19) United States
(12) Patent Application Publication
(10) Pub. No.: US 2004/0219602 A1
(43) Pub. Date: Nov. 4, 2004

(54) Evaluating effects of exposure conditions on drug samples over time

(75) Inventors: Eric D. Carlson, Cupertino, CA (US); Peter J. Desrosiers, Santa Clara, CA (US); Li Song, Santa Clara, CA (US)

Correspondence Address: SYMYX TECHNOLOGIES INC, LEGAL DEPARTMENT, 3100 CENTRAL EXPRESS, SANTA CLARA, CA, 95051

(73) Assignee: Symyx Technologies, Inc., Santa Clara, CA (US)

(21) Appl. No.: 10/790,956

(22) Filed: Mar. 1, 2004

Related U.S. Application Data
(60) Provisional application No. 60/451,463, filed on Mar. 1, 2003.

(51) Int. Cl. G01N 33/53; G01N 33/543

(52) U.S. Cl. 435/7.1; 436/518

(57) ABSTRACT

The present invention relates to the field of research for drug formulations. More particularly, this invention is directed toward an apparatus and method of performing parallel synthesis and screening of multiple drug compositions for stability and compatibility when exposed to various conditions over time, including various environmental and/or chemical conditions. In one aspect, the invention includes a method of testing for effects of a condition on a drug sample. The method includes providing an array of drug samples, simultaneously exposing a first plurality of the drug samples in the array to a first controlled condition for a period of time within an exposure period, and evaluating a plurality of the exposed drug samples at a first time and a second time using a non-destructive test in order to determine whether the condition has an affect on one or more of the drug samples over time. At least a portion of the exposure period between said first and second time, and the non-destructive test is the same each time.

### CROSS-REFERENCE TO A RELATED PATENT APPLICATION

This application claims the benefit of U.S. Provisional Application No. 60/451,463, filed Mar. 1, 2003, the disclosure of which is hereby incorporated by reference in its entirety.

### TECHNICAL FIELD

The present invention relates to the field of research for drug formulations. More particularly, this invention is directed toward an apparatus and method for performing parallel synthesis and screening of multiple drug samples for stability and compatibility when exposed to various conditions over time, including various environmental and/or chemical conditions.

### BACKGROUND

Combinatorial chemistry has revolutionized the process of drug discovery and development. See, for example, 29 Acc. Chem. Res. 1-170 (1996); 97 Chem. Rev. 349-509 (1997); S. Borman, Chem. Eng. News 43-62 (Feb. 24, 1997); A. M. Thayer, Chem. Eng. News 57-64 (Feb. 12, 1996); N. Teiret, 1 Drug Discovery Today 402 (1996)). Because of its success in eliminating the bottleneck in drug discovery, the bottleneck in getting a drug to market has simply moved to a different area of the drug-marketing pipeline. Two such bottlenecks are the evaluation of the chemical stability of drug candidates and the evaluation of compatibility of various components of drug compositions over time and under various exposure conditions.

The current drug development process occurs in several steps. Generally, development takes the following form: first, drug candidates, or active pharmaceutical ingredients, are discovered. Next, the newly discovered compound is evaluated for its biological effects, such as toxicity, administration, distribution, metabolism and excretion (ADME/Tox). These tests indicate what happens to a drug when it is introduced into the body. Next is the pre-formulation stage where several properties of the drug candidate are evaluated, such as different polymorph forms, salt selection, as well as the stability of the compound when subjected to various chemical environments, such as acidic, basic, or oxidative conditions, to name a few. Some companies perform these activities in a “preformulation” stage and pass it on to formulators along with other formulation information. The fourth step is typically the formulation stage, where the drug candidate is combined with various components, such as excipients, in order to discover a final drug composition that has the desired biological effects and can be administered in a safe and easy manner. This step includes evaluating the compatibility of the components of the drug compositions over time. These evaluations include subjecting the compositions to various environments, such as various temperatures, humidity levels and light. These evaluations indicate which compositions are the most stable over time. Finally, clinical trials are conducted, where the drug compositions from the formulation stage are administered to test subjects to evaluate actual effects of the drug composition.

The current drug development process has several limitations. One such limitation is that the evaluation of the chemical stability of drug candidates and the evaluation of the compatibility of components of drug compositions are effected individually as separate samples in separate sets of experiments and such evaluations are tedious and time consuming. These evaluations occur over a long time period, are labor intensive, and can require hundreds to thousands of experiments and require the use of larger sample sizes.

First, since the evaluation of interest is for the stability or compatibility over time, the samples must be tested over a period of time, which can be days or months. Since typically stability or compatibility evaluations are conducted at different points in the development process, the evaluations are conducted as separate tests, and in some cases are conducted by separate research groups, each requiring large amounts of time.

Second, since many different variables are to be tested (e.g., different polymorph forms, different environmental exposures, different chemical exposures, and combination of different components) hundreds to thousands of samples are required for thorough, systematic investigation. The current process is not systematic. Designs are usually based on the experience of the scientists designing the experiments. As such, many potential drug compositions are missed. Also, preparing the samples as well as testing them can be extremely time consuming. For example, typically, each sample is measured out by hand and transferred from preparation areas to exposure areas to testing areas. When grams, or in some cases, micrograms, of drug candidate are used, the measured amounts can be extremely inconsistent from one sample to another, and transferring samples can lead to loss of some of the sample, which in a microgram case, can be extremely relevant. The entire preparation process is also time consuming.

Third, the samples that are subjected to the various conditions are typically tested using a destructive test (a test that destroys the sample, or portion of the sample tested), such as HPLC, in order to determine if any change has occurred in the sample. If it is desired to test the sample at several different points in time during the test in order to generate a time dependant curve of any change, a large enough sample is required so that a portion of the sample can be removed to be tested and destroyed each time a data point is needed, or enough replicate samples need to be created at the outset to provide for the intended tests. This can become a problem if there is a limited amount of drug candidate available for evaluation. Also, in those situations where replicate samples are exposed, and different samples are removed from exposures at different times in order to generate time dependent exposure data, the tests are conducted on different samples each time. While this can be acceptable, it is not ideal. A more ideal situation would be to test the same sample at different times throughout the exposure to generate time dependent exposure data for the exact same sample.

Finally, samples and portions of samples are often transferred from one location to another for exposures and testing. When this occurs, the samples are typically moved from one substrate or vessel to another. This can result in the loss of sample through handling error. While the loss is typically small, this is still a problem when a limited amount of drug candidate is available.

As is known in the pharmaceutical industry, environmental conditions, such as various levels of temperature, humidity and light, as well as chemical exposure, such as acidic or basic exposure, when occurring over a period of time, can have an adverse affect on the stability of an active drug ingredient, as well as on the compatibility of various components of drug compositions. This necessitates the evaluation of the shelf life of drug candidates as well as drug compositions containing various components. Excipients can have functional groups that interact directly with drug candidates, or can contain impurities or residues, or form degradation products that cause decomposition of the drug candidate.

Several published patent applications in the area of high throughput or combinatorial materials science disclose a workflow where the materials created in the workflow can be screened on the same plate in which they are synthesized. For example, WO 99/59716 discloses and claims creating solids on a removable reactor base plate and then performing X-ray analysis of the solids. WO 01/34290 and WO 01/34291 reportedly relate to a “work station” that employs an array that can be transferred between synthesis, screening and characterization stations without requiring sample handling, preparation or transfer steps. See also U.S. Pat. Nos. 6,371,640 and 6,004,617, which are incorporated by reference. In addition, WO 96/11878 discloses parallel crystallization and screening of materials on a substrate. Also, WO 01/51919 reports a high throughput method for formation, identification and analysis of diverse solid-forms.

A need generally exists in industry for a combinatorial or high throughput method and apparatus for the research, discovery and development of drug compositions. This need has developed due to the ever-increasing number of active compounds discovered by high throughput discovery efforts in the pharmaceutical industry.

Despite the cited work, a workflow for the systematic high throughput research of the chemical stability of drug candidates and component compatibility of drug composition components has not been directly disclosed. Thus, this invention provides a universal system that solves, at least in part, this need, beginning with library design and ending with evaluations for performance, with a variety of designing and evaluation options.

### SUMMARY OF THE INVENTION

Chemical stability of drug candidates and excipient compatibility are key steps in the drug development process. In general, this invention provides an integrated, automated workflow and system that allows both chemical stability and excipient compatibility studies to be executed in a parallel and automated fashion that uses much smaller amounts of material than conventional studies and covers a much broader range of potential exposure conditions. The integrated hardware and software used to execute this workflow will be described below and is complimentary to the Symyx Discovery Tools® Polymorph System described in U.S. patent application Ser. No. 10/156,329 (Publication No. 2003-0118078), U.S. patent application Ser. No. 10/156,245 (Publication No. 2003-0116497), U.S. patent application Ser. No. 10/156,222 (Publication No. 2003-0124028) and U.S. patent application Ser. No. 10/156,295 (Publication No. 2003-0119060) which are hereby incorporated by reference in their entirety.

In one aspect, the invention provides a three part methodology or workflow, which includes: (1) creation of a library of drug candidates and/or compositions (drug samples), (2) exposing the library to an environmental and/or chemical condition, and (3) evaluating the library for any chemical or physical change over time when exposed to the condition(s). This methodology accomplishes the task of evaluating drug candidates for stability over time under various exposure conditions, as well as evaluating the compatibility of drug composition components over time under various exposure conditions. These tasks can be accomplished in several ways as is discussed below. The stability of drug candidates can be evaluated separately from the drug composition component compatibility evaluation, or the two can be evaluated together in a common set of samples using a common set of experiments.

In another aspect, the invention provides a system capable of formulating arrays of both solid and liquid samples, exposing the arrays to various environmental and/or chemical stresses, and rapidly analyzing the samples for physical and/or chemical stability, for potency, and/or for the appearance of degradation product.

In another aspect, the invention provides methods in which stability studies are carried out to determine the chemical stability of a drug candidate, its compatibility with potential excipients, and its stability in various formulations. In one embodiment, a stability study begins with a library design prepared using Library Studio® software (Symyx Technologies, Santa Clara, Calif.), and is intended to reveal the effects of acids, bases, oxidants, radical generators, temperature, relative humidity, and light intensity on the potency of an active ingredient of a drug candidate. An alternative design explores the excipient compatibility for liquid or solid formulations. Additional designs that focus on co-solvent polarity, co-solvent viscosity, complexing agents, dispersants, surfactants, pH, or combinations of stabilizers can also be employed depending on the active compound of interest.

In one aspect, liquid samples (solutions, emulsions, and dispersions) are created on a sample preparation station employing appropriate software, such as Impressionist® software available from Symyx Technologies (Santa Clara, Calif.). In general a solutions of drug candidates in a volatile solvent (e.g. dichloromethane, methanol, or acetonitrile) are made and dispensed uniformly into an 8×12 array of 1 mL glass vials. The solvent is then removed by evaporation. Alternatively, the drug candidate may be dispensed as a solid powder. Stock solutions of the other components (e.g. HCl, NaOH, HOOH, AIBN, etc.) are dispensed to the array of vials. The vials are sealed and samples are agitated and allowed to dissolve to room temperature solution equilibrium. The vials can be sealed with common or individual septa or lids that are adapted for subsequent operations. In one embodiment in which a piercable septum is employed, a robotic pH probe with a piercing needle is then used to measure the pH of each sealed sample. In one aspect, replicate plates are made for each of the environmental conditions being tested. Each sealed array of vials of liquid samples is placed into an environmental chamber and exposed to various heat and light conditions.

In another aspect, arrays of solid samples are prepared by one of several methods. Crude solid formulations are prepared by dispensing solutions of components (drug candidate, mannitols, sorbitols, povidones, tweens, etc.) to an array of wells with the sample preparation station and evaporating off the solvents. Robotic methods and apparatus deliver and mix in place small quantities of solid powders directly into the array of wells. Solid formulations are dispensed either into an 8×12 array of 1 mL glass vials or into a solids isolator block made to house a common universal substrate that allows birefringence, Raman, powder XRD, and other analyses to be performed without further manipulation of the solid formulations off of the substrate. Replicate plates can be made for each of the environmental conditions being tested. Each array of samples is placed into an environmental chamber and exposed to various heat, humidity, atmospheric and light conditions.

In another aspect, each array of samples is placed into an environmental chamber to accelerate the potential degradation of samples under a variety of environmental conditions. Samples can be logged into and out of the various environmental chambers with bar code readers so that the environmental history of each sample will be automatically recorded and stored to the database. Sealed vials of the samples are exposed to heat (for example, 55° C., 70° C., and 85° C.), light exposure in a photo stability chamber, and room temperature. Open arrays of solid samples (which can be pressed into tablet form, preferably in parallel) are exposed to heat and humidity (for example, 40° C./80% RH, 25° C./60% RH, 70° C./4% RH), light exposure in a photo stability chamber, and room temperature.

In one aspect, automated pH readings are taken of each well using a pH probe mounted on a robotic arm. The probe tip is immersed in the sample fluid and a series of measurements are taken until the reading is stable. The tip is subsequently washed before dipping into the next sample. pH readings are automatically collected and written to a database.

In another aspect, liquid chromatography, such as high performance liquid chromatography (HPLC), which can be performed in a rapid serial or parallel mode, is used to quantify the potency of the drug candidate. The drug candidate is rapidly separated from the various excipients and the drug candidate's concentration is quantified. A limited set of conditions is also employed to separate and identify the presence of decomposition products as they emerge. Software, such as Epoch™ software available from Symyx Technologies, is used to control the dilution of the aliquots, LC data acquisition, and data storage. The LC method, LC data, retention times, and peak areas, for each measurement are stored in a database, such as those available from Oracle. The data are automatically analyzed for potency versus time or % increase in decomposition product versus time. When possible, estimates are made of reaction order along with rate constants and activation energies.

In another aspect, aliquots of liquid samples are drawn from a sealed array of liquid formulations using a needle assembly on the sample preparation station. Samples can be optionally filtered prior to sampling with a robotic parallel filtration assembly that allows samples to be removed from heated, sealed vials, filtered, and transferred to a heated, sealed plate while maintaining the sample temperature. Aliquots can be automatically diluted for HPLC analysis. The workflow enables plates to be robotically logged out of the environmental chambers, sampled, and logged back into the environmental chamber for continued environmental exposure.

Another aspect of the invention provides for the dissolution and dilution of solid samples in an appropriate solvent to be analyzed using UPLC. If the samples are contained in an 8×12 array of glass vials they can be dissolved in place. If the samples are held on a common planar, wafer-type substrate, the substrate can be resealed to a solids isolation assembly block and samples are dissolved in place. The workflow enables plates to be robotically logged out of the environmental chambers, sampled, and logged back into the environmental chamber for continued environmental exposure.

Another aspect of the invention provides for the use of rapid, non-destructive, non-contact screening techniques to avoid the excessive liquid handling that destructive tests, such as LC analysis, require and to reduce the number of daughter plates being stored in the environmental chambers. The rapid analytical station relies on a spectroscopic method such as Raman, FT-Raman, or near-IR spectroscopy, for example. The Raman station can be a commercial Raman spectrometer specifically modified to run library arrays controlled by software. Samples are positioned (?) on a substrate that is mounted on an XYZ stage. Software translates the sample array from sample to sample, and collects the spectrum and an optical image of each sample, and stores the data in the database. Changes in peak heights from plate to plate are quantified and the spectra are sorted into groups to identify the presence of decomposition products.

Another aspect of the invention provides for tracking the solid form of a drug candidate by using X-ray diffraction (XRD) as a non-contact, non-destructive, rapid analytical screen. The XRD station can be a reflective XRD diffractometer with an XYZ stage. Software controls the automated positioning and acquisition of XRD patterns for each of the samples and stores the data in the database. XRD collection time for samples as small as 0.25 mg can be less than five minutes. Changes in form are identified and a screening estimate of the overall crystallinity of the sample can be provided.

Another aspect of the invention provides for evaluation of the dissolution rate of solid formulations using a rapid dissolution station. An array of solid samples is placed on the deck of the station, and a robotic tip dispenses dissolution media with agitation. A second tip then takes small, filtered aliquots over time that are analyzed by rapid HPLC. The evaluation is useful to consider formulations that contain from 0.01-0.5 mg drug candidate dissolving into 1-10 mL of dissolution media.

Another aspect of the invention provides a method of testing the compatibility of components of a drug composition over time when exposed to a condition. The method includes providing an array of drug compositions and exposing a first plurality of the drug compositions in the array to a first condition for a period of time within an exposure period. The condition can be environmental or chemical. Environmental conditions can include, for example, various temperatures, light exposures, humidity levels, and atmospheres. Chemical conditions can include, for example, acidic, basic, oxidative and radical producing environments. A plurality of the exposed drug compositions are tested at least twice using a non-destructive test in order to determine an effect of the first condition on one or more of the drug compositions or components thereof over time. At least a portion of the exposure period is between the two tests. The drug compositions can be tested multiple times during the exposure period. Testing the drug samples multiple times throughout the exposure period gives a time dependant analysis of any change in the drug compositions. Testing the drug samples with a non-destructive test (a test that does not consume the sample) allows the original sample to be smaller, because the same sample can be tested multiple times without having to destroy a portion of it.

Another aspect of the invention provides a method of testing the effects of an exposure over time on the stability of a drug candidate. The method includes providing an array of drug candidates and exposing a first plurality of the drug candidates in the array to a condition for a period of time within an exposure period. The exposure condition can be environmental or chemical. A plurality of the exposed drug candidates are tested at least twice using a non-destructive test in order to determine an effect of the condition on the chemical or physical stability of one or more of the drug candidates over time. At least a portion of the exposure period is between the two tests.

Another aspect of the invention provides a method for evaluating the compatibility of drug composition components when exposed to various conditions over time. The method comprises providing an array of drug compositions, and simultaneously exposing a plurality of the drug compositions to at least one environmental condition for a period of time within an exposure period. A plurality of the drug compositions, which can be the same or different than the plurality exposed to the environmental condition, are simultaneously exposed to at least one chemical condition for a period of time within the exposure period. The exposed drug compositions are then evaluated for any chemical or physical change. The evaluation can occur at one time or multiple times, for example evaluated before, during and/or after the exposure period.

Another aspect of the invention provides a method for evaluating the stability of drug candidates when exposed to various conditions over time. The method comprises providing an array of drug candidates, and simultaneously exposing a plurality of the drug candidates to at least one environmental condition for a period of time within an exposure period. A plurality of the drug candidates, which can be the same or different than the plurality exposed to the environmental condition, are simultaneously exposed to at least one chemical condition for a period of time within the exposure period. The exposed drug candidates are then evaluated for any chemical or physical change. The evaluation can occur at one time or multiple times, for example evaluated before, during and/or after the exposure period.

Another aspect of the invention provides a process for developing a drug composition. The process comprises evaluating an array of drug compositions and drug candidates for the effects of an exposure condition over time on the stability of one or more drug candidates and the compatibility of one or more drug composition's components using a common set of samples in a common set of experiments. The process also includes formulating a clinical sample comprising a screened drug composition and evaluating the formulated clinical sample in one or more clinical trials.

Another aspect of the invention provides a method for testing the compatibility of components of a drug composition over time under exposure conditions. The method includes providing a drug composition sample comprising less than 40 mg of drug candidate, exposing the sample to at least one condition, which can be environmental or chemical, for a period of time within an exposure period, and generating data from at least one non-destructive test conducted on the sample at least twice to determine if there has been any physical or chemical change in the sample. At least a portion of the exposure period is between the two tests.

Another aspect of the invention provides a method for testing the possible effects of a condition on the stability of a drug candidate over time. The method includes providing a drug candidate sample weighing less than 40 mg, and exposing the sample to at least one condition for a period of time within an exposure period. Data is generated from at least one type of non-destructive test conducted on the sample at least twice to determine if there has been any physical or chemical change in the sample. At least a portion of the exposure period is between the two tests.

Another aspect of the invention provides a method for evaluating the possible effects of a condition on a drug candidate or a drug composition. The method includes providing an array of drug compositions, drug candidates, or a combination thereof on a single substrate and exposing the array and the substrate to at least one condition for a period of time defining an exposure period. Data is generated from at least one test conducted on the array at least twice to determine if there have been any changes in the samples of the array during the exposure period. The array remains on the substrate throughout the generating step, and at least a portion of the exposure period is between the two tests. The exposure can be environmental, chemical, or a combination.

Another aspect of the invention provides a method for testing a drug composition or drug candidate sample. The method includes providing the drug composition or drug candidate and simultaneously exposing the sample to a controlled light condition and at least one of a controlled humidity and temperature condition in an environmental chamber for an exposure period. The sample is tested, and the effects of the exposure over time on the sample are evaluated.

Another aspect of the invention provides a method for generating data for analyzing the chemical changes of a drug composition over time. The method includes providing five arrays of drug composition samples, drug candidate samples, or combinations thereof, and simultaneously exposing the five arrays to five different environmental conditions for an exposure period. The first array of drug composition samples is exposed to a first controlled temperature setting, the second array is exposed to a second controlled temperature setting, the third array is exposed to a first controlled humidity setting, the fourth array is exposed to a second controlled humidity setting, and the fifth array is exposed to a controlled light setting. All of the arrays are tested with a non-destructive test at least twice with at least a portion of the exposure period being between the two tests. Data is generated from the non-destructive tests for each array of drug compositions to determine the compatibility of drug composition components, or the stability of drug candidates over time and with respect to various environmental exposure conditions.

In some embodiments, the invention features techniques for performing the combinatorial or high throughput analysis of libraries of drug candidates and compositions. These techniques can be implemented to decrease the time needed to evaluate the chemical stability of a variety of drug candidates and the compatibility of drug composition components when exposed to environmental and/or chemical conditions over time. These techniques also allow for additional drug compositions to be discovered, possibly allowing for additional patent coverage, decreased risk of competitors discovering a related formulation and decreased risk of unwanted drug compositions appearing in later stages of pharmaceutical development. In addition, the techniques disclosed herein allows for multiple different drug compositions to be simultaneously exposed to chemical conditions and environmental conditions and tested in parallel, thereby creating a high throughput methodology for pharmaceutical research organizations and others.

Thus, a method is provided for testing drug candidates and drug compositions, which includes subjecting to environmental and/or chemical conditions, in parallel, one or more drug candidates or compositions residing on a substrate. The drug samples can include different components, but can include the same drug candidate. The drug samples, which can be in any form, such as solid or liquid, reside on or in the substrate, typically in regions so that the compositions can be tested individually. The method further provides for the option of testing the samples for at least any structural change while the samples are exposed to environmental and/or chemical conditions while residing on the substrate.

Another aspect of the invention provides a system for parallel evaluation of drug samples, including: an array of, for example, 8 members in regions of an optically transparent glass substrate. The members in the regions are isolated from one another such that they do not or cannot mix, and each of the members includes one drug candidate. The system also includes a temperature controlled block for holding the substrate, the block having holes there-through corresponding to the regions of the substrate, such that radiation can pass through the block and the regions of the substrate.

Another aspect of the invention provides software that can track sample history, organize test data and sort for and recognize changes (i.e., appearance of new peaks or change in peak intensity) to show the time dependent change in the samples during exposure.

Another aspect of the invention provides an apparatus for synthesizing and testing arrays of drug samples for evaluation-. The apparatus includes a sample preparation station, an environmental exposure station and a rapid analytical station. In other aspects of this invention, one or more of the assemblies is provided separately (e.g., the sample preparation assembly).

The various aspects of this invention can be combined into a flexible workflow that includes a sample preparation station (e.g., an assembly for combining starting materials), a daughtering station for optionally daughtering the libraries, exposure station(s) for exposing the libraries to environmental and/or chemical conditions and testing station(s) for testing the libraries during and after exposure. In one aspect, a single substrate is used for the testing protocols, particularly those testing protocols that involve optical techniques, such as birefringence, Raman, and X-ray diffraction.

The techniques described herein can be implemented to provide for the rapid creation and testing of drug samples, and offers significant advantages over conventional experimental methods and systems. For example, some of the methods and apparatus described herein allow for automated parallel drug sample creation, automated parallel drug sample exposure and automated parallel drug sample evaluation, thereby saving time and conserving valuable drug candidate in determining appropriate drug compositions for formulation studies. Other aspects include a variety of evaluation options, allowing for flexibility in choosing the appropriate technique for monitoring a change in the drug samples over time.

Thus, the flexibility of this invention includes a variety of options that a complete system offers, including choosing starting components (e.g., drug candidates and excipients), choosing exposure conditions for the drug samples and choosing characterization methods and apparatus.

The invention can be implemented to provide one or more of the following advantages. One advantage of the invention to prepare and evaluate drug samples in parallel, for example, in microtiter plate format, using between 0.01 and 40 mg of drug candidate per region or well. Another advantage of the invention is evaluating the time dependent effects of environmental and chemical conditions on libraries of drug samples in order to understand the stability of the drug candidates or compatibility of the composition components. Another advantage of the invention is to non-destructively test drug samples that are being exposed to a condition or set of conditions at various times before, during and/or after exposure in order to evaluate any time dependent change in the samples. Another advantage of the invention is to evaluate the compatibility of a large variety of excipients with different drug candidates over time when exposed to a condition to determine optimal components for drug formulation development. Another advantage of the invention is to simultaneously expose an array of drug samples to environmental and chemical conditions in order to evaluate the stability of drug candidates and the compatibility of drug composition components using a common set of tests on a common set of samples. Another advantage of the invention is to use automation (robotics and software) to perform testing of drug samples in a rapid serial or parallel manner and to use software to determine the stability and/or compatibility change over time of the drug samples. Another advantage of the invention is to use a single substrate for non-destructive test measurements to be made on the drug samples without transfer of material.

It is considered and understood that the many features and aspects of the embodiments described herein can be combined with each other.

A further understanding of the nature and advantages of the present invention can be realized by reference to the remaining portions of the specification and the drawings.

### BRIEF DESCRIPTION OF THE DRAWINGS

FIG. 1 is a flow diagram showing a general method for the time dependent stability evaluation of drug candidates, the compatibility evaluation of drug composition components and combinations over time under exposure conditions.

FIG. 2 is a flow diagram depicting a method for one embodiment of creating libraries for the present invention.

FIG. 3 shows a block diagram of a workflow for one embodiment of a methodology useful in this invention.

FIG. 4 is a cut-away perspective illustration of a glass lined vessel container useful in this invention.

FIG. 5 includes FIGS. 5A-5C and shows details of a library storage rack and plate system useful in this invention.

FIG. 6 shows one embodiment of a daughtering station or sample preparation station that can be used in the present invention.

FIG. 7 shows an overhead view of an embodiment for an automated apparatus that can be used in the present invention for evaluating large numbers of drug samples for time dependent changes under various controlled exposure conditions.

FIG. 8 consists of FIGS. 8A, 8B, 8C, 8D and 8E and shows example library designs that can be used in the present invention.

FIG. 9 consists of FIGS. 9A, 9B, 9C, and 9D and shows Fluorescence decomposition data for Amoxicillin, Ampicillin trihydrate and Cephalexin samples that have been exposed to a controlled temperature as well as fresh samples as described in Example 1.

FIG. 10 consists of FIGS. 10A, 10B and 10C which show Raman decomposition data for Amoxicillin, Ampicillin trihydrate and Cephalexin samples that have been exposed to a controlled temperature as well as fresh samples as described in Example 2.

FIG. 11 shows X-ray powder diffraction decomposition data for Amoxicillin that has been exposed to a controlled temperature as described in Example 3.

FIG. 12A shows Fourier Transform Near—IR decomposition data for Amoxicillin that has been exposed to a controlled temperature as described in Example 4.

FIG. 12B shows Fourier Transform Near—IR decomposition data for Cephalexin samples that have been exposed to a controlled temperature as described in Example 4.

FIG. 13 shows Fourier Transform Mid-IR decomposition data for Amoxicillin that has been exposed to a controlled temperature as described in Example 5.

FIG. 14 shows UV-Vis diff-use reflectance decomposition data for Amoxicillin that has been exposed to a controlled temperature as described in Example 6.

FIG. 15A shows colorimetry data for Amoxicillin samples that have been exposed to a controlled temperature as described in Example 7.

FIG. 15B shows colorimetry date for Armoxicillin samples that have been hydrolyzed to a pre-determined amount as described in Example 7.

### DETAILED DESCRIPTION

The invention provides techniques for investigating drug candidates for chemical and physical stability and compatibility with other components over time. In general, a methodology or workflow for performing such investigations includes: (1) the creation of a library of drug candidates and/or compositions (drug samples); (2) exposing the library to an environmental and/or chemical condition; and (3) evaluating the library for any chemical or physical change over time when exposed to the condition(s). A chemical change occurs when a chemical bond is broken or formed, and a physical change occurs when the physical state of the sample changes, such as phase separation or a change in morphology, but the chemical bond structure remains the same.

This methodology can be implemented to accomplish the task of evaluating drug candidates for stability over time under various exposure conditions, as well as evaluating the compatibility of drug composition components over time under various exposure conditions. These evaluations can be performed in several ways, as is discussed below. The stability of drug candidates can be evaluated separately from the drug composition component compatibility evaluation, or alternatively, the two can be accomplished together in a common set of samples using a common set of experiments.

As discussed herein, a drug candidate is a compound (salt or neutral) shown to have pharmacological (prophylactic or therapeutic) activity. A typical drug candidate is a small molecule (as opposed to a protein), but the exact nature of the compound is not critical to this invention, and the drug candidate need not have been proven to be safe and/or effective for use in any therapeutic application. Some drug candidates have salts that are anionic or cationic and some drug candidates are neutrals. No matter the form, drug candidates can have different crystallographic polymorphs. Herein, the term “polymorph” is intended to include polymorphs, pseudo-polymorphs, hydrates, solvates and the like.

As used herein, the term “drug composition” refers to a composition that has one or more drug candidates and at least one excipient. A drug composition can take any form, such as a liquid, solid, solution, suspension, dispersion (uniform or non-uniform), solid-liquid emulsion or a liquid-liquid emulsion and can include solvents and co-solvents.

In addition, as used herein, the term “excipient” refers to a drug composition component that is typically intended to aid in manufacture, administration, absorption, appearance enhancement or retention of quality of a drug. Excipients rarely, if ever, possess pharmacological activity by themselves, and are accordingly loosely characterized as being substantially “inert.” However, excipients can initiate, propagate or participate in chemical or physical interactions with a drug candidate, possibly leading to compromised or enhanced quality or performance of the drug. One example of an excipient that is commonly used is a solvent. For example, solvents can have an effect on the reaction rate of a drug candidate, or the degradation rate of a drug can change with the dielectric constant of the medium. As a specific example, certain studies have shown an increase in photo stability of Vitamin-B12 by the addition of viscogens such as glycerol or Ficoll. See Rong, Lui (editor), Water-Insoluble Drug Formulation, Chapter 7 “Solubilizatinon Using CoSolvent Approach”, J. Trivedi and M. Wells (authors), Interpharm Press, 2000, pp. 141-168, which is hereby incorporated by reference.

A “sample” refers to a single unit that is being evaluated. A “drug sample” refers to a sample that is a drug candidate, a combination of drug candidates, or a drug composition.

An “exposure period” refers to the period of time between the beginning of the exposure and the final removal of the sample from the exposure during which a sample or array of samples is exposed to a condition. The sample(s) can be continuously or intermittently exposed to the condition. The exposure period begins when the sample(s) is/are initially exposed to a condition, and the exposure period ends when the sample is removed from the exposure condition with the intent of terminating the exposure. The exposure time is the total amount of accumulated time of exposure, and the exposure period is the period of time between the beginning of exposure and final removal of the sample from the exposure condition. In situations in which the sample(s) is/are removed from the exposure condition for testing, then placed back in the exposure condition, the exposure time can represent the sum of the sub-periods of exposure but typically does not include the time during which the samples are not exposed to the condition.

An “array” refers to a collected association of at least two samples. The samples of the array can be the same samples or different samples. Different samples can differ in chemical composition, such as samples that include different drug candidates, different excipients, different reactive chemicals, and different polymorph structures. Samples of an array can also differ by relative ratios and/or concentrations of components.

The techniques described herein can involve the formation of libraries of drug candidates and/or drug compositions. As used herein the term “library” refers to a combination of a plurality of materials in discrete regions of a common substrate. The materials in the library can be the same or different. Libraries for use in these techniques can be designed based on the principles discussed herein, and library design software, such as Library Studio™ (available from Symyx Technologies, Inc., Santa Clara, Calif.), can be employed to set out the drug candidate type, drug candidate form, salt(s), excipient(s), exposure chemicals, etc. to be employed. Library Studio is described in WO 00/23921 and EP 1080435, which are herein incorporated by reference.

In certain aspects of the invention, a library can have composition diversity, exposure diversity, or both. Composition diversity refers to a variance in the composition or structure of library members. These members can comprise different drug candidate numbers, amounts, types, crystal(s), salt(s) or polymorphs of a drug candidate, the number, type and amount of excipients that are used to formulate drug compositions, as well as different amounts or ratios of components, etc. For example, a library can include different polymorphs of a drug candidate in a plurality of wells, or different drug candidate types in a plurality of wells, or any combination of different drug candidates, their polymorphs and excipients, etc. Exposure diversity refers to a variance in the exposure conditions, such as differing levels of light, temperature, humidity, acidity, alkalinity, oxidation, reduction, etc., to which the members of a library are exposed (where the members may or may not have the same composition or structure). The members in the library can have varying amounts (volume, moles or mass) and ratios of components, and other conditions that those of skill in the art will recognize. It is through the creation of libraries having diversity and the screening of that diversity that a complete combinatorial research and development program can be undertaken for evaluating the stability of drug candidates and the compatibility of drug composition components over time.

Libraries can also incorporate chemical and/or physical diversity. Chemical diversity refers to a difference in the chemical formulae of two or more members of a library, while physical diversity refers to a difference in physical features (even though the library members can have the same chemical makeup). Physical diversity can occur, for example, when there exist a plurality of crystalline or polymorph forms of a single drug candidate. There can be physical diversity between drug candidate members, for example, in a library that includes various polymorph forms of the same drug candidate.

In particular embodiments, the techniques described herein can be implemented with four types of libraries: drug candidate libraries, excipient libraries, drug composition libraries and drug sample libraries. Individual libraries can be combined with or into other libraries and/or used together in the various aspects of the invention.

A drug candidate library is a library comprised of drug candidates. Typically, a drug candidate library has chemical and/or physical diversity. In some embodiments, a plurality of members in the library can be the same drug candidate, both physically and chemically. Such a library can be used, for example, when reproducibility of results is desired. A drug candidate library includes at least one drug candidate, although 2, 3, 4, 5 or more different drug candidates can be present in a single drug candidate library. The number of different drug candidates and the number of different physical forms of the drug candidates will depend on the specific application.

There are many different combinations possible in a drug candidate library, depending on the desired evaluation. Thus, a drug candidate library can include different drug candidate members in different columns, and/or can have different polymorph forms in different rows for example. The drug candidate library members can include different drug candidates or can all include different physical forms of one drug candidate. Also, the drug candidate library can have two or more members that are identical as a redundancy option, when exposure conditions are to be varied, or when the drug candidate library is to be combined with other libraries or components, for example. The drug candidates in the drug candidate library can exist in many forms, such as solids, liquids, solutions, emulsions, or dispersions. When a solid phase drug candidate library is to be evaluated or used to create drug composition libraries, the solid member drug candidates can be dissolved in a suitable solvent to allow for liquid handling.

An excipient library is a collection of drug excipients. Excipients can take the form of a solid, liquid, dispersion, or emulsion. An excipient library includes one or more types of excipients, provided in one or more forms, and can be arrayed or designed to have a logical order. For example, one excipient library can include one specific type of excipient, such as lactose. In other embodiments, the library can be organized to include columns of similar excipients with each row having diversity. For example, the library can have 96 wells having 12 columns and eight rows. A library of lubricating excipients, for example, can be provided with each column containing a different type of lubricant discussed above for drug candidate libraries.

In one embodiment, an excipient array is based in a 96 well microtiter plate and has up to 96 different excipients. Since a 96 well microtiter plate has eight rows and twelve columns, there are literally thousands of possibilities. As an example, four of the rows can contain water, while four of the rows contain glycerin. Each row can also have a different secondary excipient that varies with the first excipient across the twelve columns from 100% to 0% in a linear gradient fashion. Thus the first column can have no first excipient and the twelfth column can have no second excipient, while the middle columns (2-11) have a mixture of first and second excipients that vary in a linear gradient fashion, keeping the total volume constant.

Drug composition libraries can include combinations of drug candidates and excipients. In some embodiments, a drug composition library can be formed from the combination of a drug candidate library and an excipient. In other embodiments, a drug composition library is formed from the combination of an excipient library and a drug candidate. In other embodiments, a drug composition library is formed from the combination of a drug candidate library and an excipient library. For example, combining can include adding at least one drug candidate member from a drug candidate library to at least one excipient. More typically, at least four drug candidate members, at least 10 drug candidate members, at least 25 drug candidate members, at least 50 drug candidate members or at least 96 drug candidate members are provided that are each combined with at least one excipient. Also, for example, combining can include adding at least one excipient member from an excipient library to at least one drug candidate. More typically, at least four excipient members, at least 10 excipient members, at least 25 excipient members, at least 50 excipient members or at least 96 excipient members are provided that are each combined with at least one drug candidate.

A number of methods can be used to combine excipients with drug candidates to form a drug composition library. In some embodiments, the same excipient is added to drug candidate members of a drug candidate library (whether the members have physical or chemical diversity or not) or different excipients are added to different drug candidate library members. In other embodiments, a different excipient is combined with each member drug candidate having a different physical form such that the number of different excipients is equal to the number of different physical forms. In still other embodiments, different excipients are added to drug candidate members having chemical diversity. Combining different excipients with different drug candidate library members provides the opportunity to evaluate the compatibility of a large amount of components. In some embodiments, drug candidate members are mixed with a suitable excipient prior to or simultaneous with allowing the drug composition(s) to be exposed to the conditions in the evaluation phase. When a drug candidate member is mixed with an excipient, a drug composition is formed.

Drug sample libraries can include drug candidates, drug compositions and combinations thereof. For example, a drug sample library can include drug candidates and drug compositions. This can be useful for simultaneously evaluating the stability of a candidate or candidates and the compatibility of the candidate or candidates with one or more excipients when exposed to a condition of interest. For example, half of the members of a drug sample library can be drug candidates, and the other half of the library can be drug compositions which contain the same drug candidates. This type of library enables the simultaneous evaluation of the stability of a drug candidate and the compatibility of one or more drug compositions using a common set of samples in a common set of experiments.

Thus, combining drug candidates and excipients provides powerful options for evaluating the chemical and physical compatibility and stability of a large variety of drug compositions over time. The use of different exposure conditions for the same or similar drug compositions and drug candidates in a combinatorial fashion can minimize the chances of missing any adverse effect the member of interest can have under certain conditions.

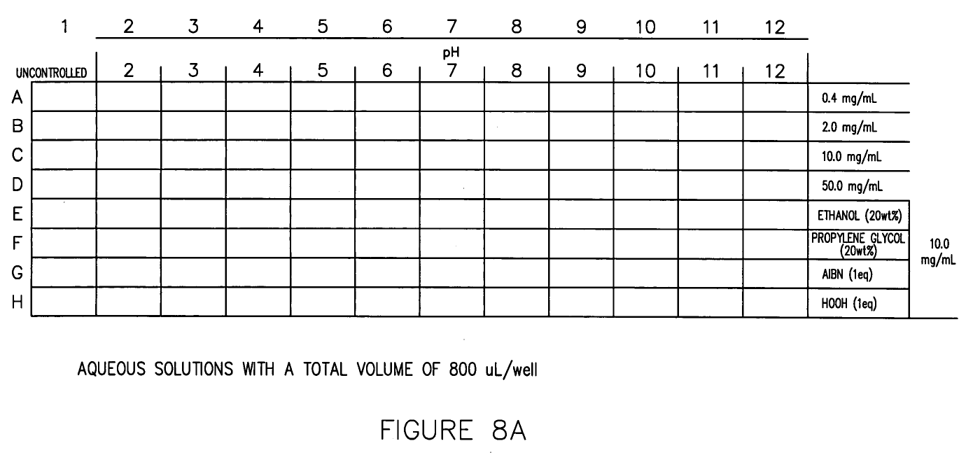

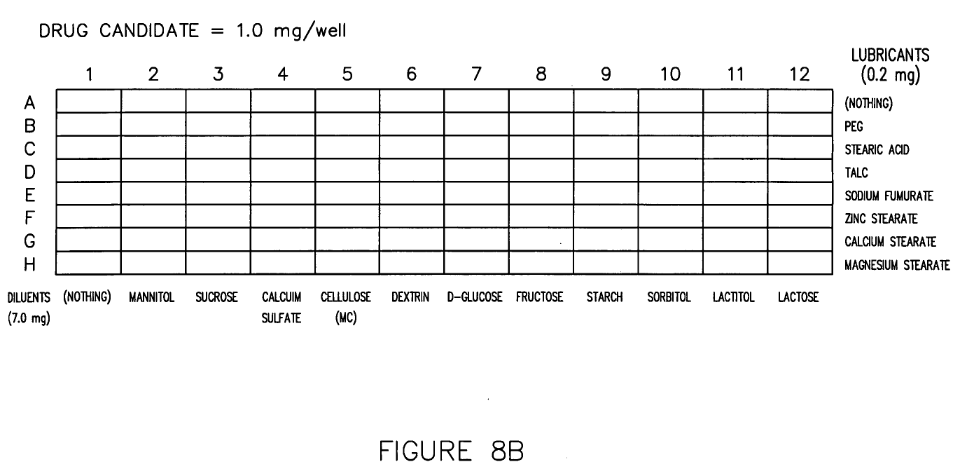

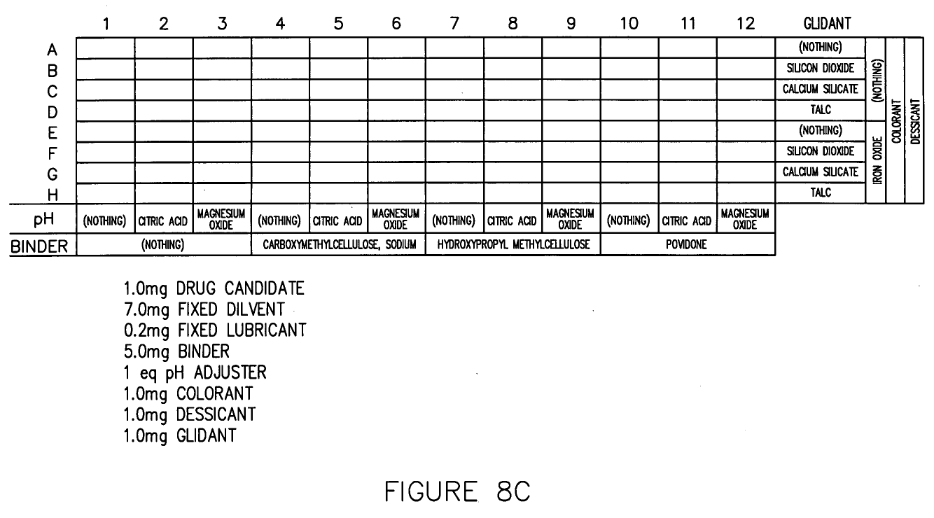

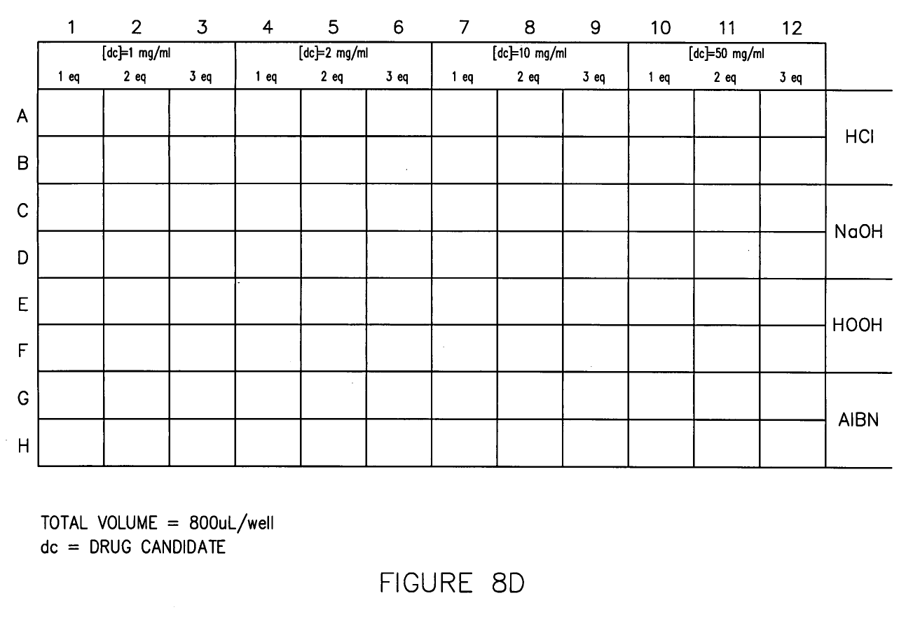

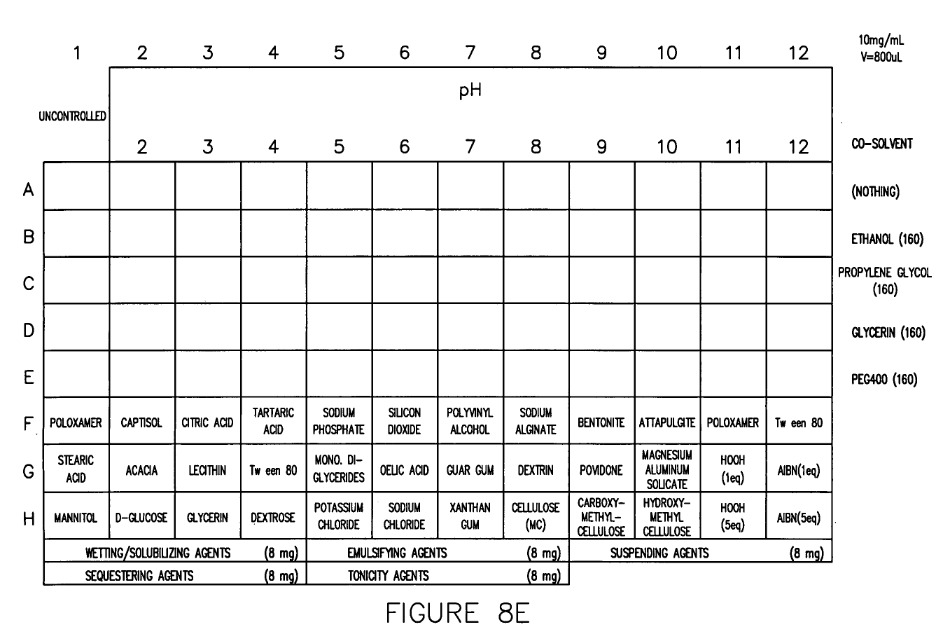

Some examples of libraries that can be used in the techniques described herein are shown in FIGS. 8A-E. In these examples, the design uses a 96 well format with 12 columns and 8 rows. FIG. 8A is an example library design for evaluating the stability and excipient compatibility of drug compositions and includes chemical exposure for the samples. The library incorporates one drug candidate which is provided in an aqueous solution in various concentrations ranging from 0.4 mg/mL up to 50.0 mg/mL as indicated in rows A-H. Rows E and F also include co-solvents that are combined with the samples, and rows G and H include the exposure of the samples to AIBN which is a radical forming compound, and HOOH which is an oxidizer.

FIG. 8B shows a library design example for evaluating different levels of excipients when combined with a drug candidate. The library is designed to use one drug candidate and combine it with several types of lubricants (rows) and several different types of diluents (columns).

FIG. 8C shows an example library useful for excipient compatibility evaluation. In one embodiment, this library could be designed after evaluation of the library in FIG. 8B. In this design, a drug candidate, a diluent and a lubricant are used throughout the library and are combined with various types and levels of binders, glidants and colorants at various pH levels.

FIG. 8D shows an example library design which varies the concentration of an aqueous drug composition across the columns and exposes them to varying types and levels of chemical species across the rows. This library is designed for every sample to be duplicated.

FIG. 8E is another example library design that can be used to evaluate the compatibility of various types and levels of excipients with a drug candidate and to evaluate the response of the excipients and drug candidates to various chemical conditions, such as pH levels, oxidizing agents and radical forming species.

FIGS. 8A-E demonstrate just a few of the design possibilities for the evaluation methods described herein, and are not intended to be limiting in any way. Various features from these library examples can be combined and used with each other.

The libraries used in the techniques described herein can be prepared or obtained in a variety of ways, and the particular method of preparation is not critical to this invention. Thus, in some embodiments libraries can be prepared as described below; prepared libraries can in some cases even be purchased from commercial sources. One or more libraries can be stored and retrieved from a storage rack for transfer to either a daughtering station, a diluting station or a dissolution station, as discussed below. Such retrieval and transfer to another station can be automated using known automation techniques, such as those disclosed in WO 98/40159, incorporated herein by reference. Robotic apparatus is commercially available, for example from Cavro, Tecan, Robbins, Labman, Bohdan or Packard.

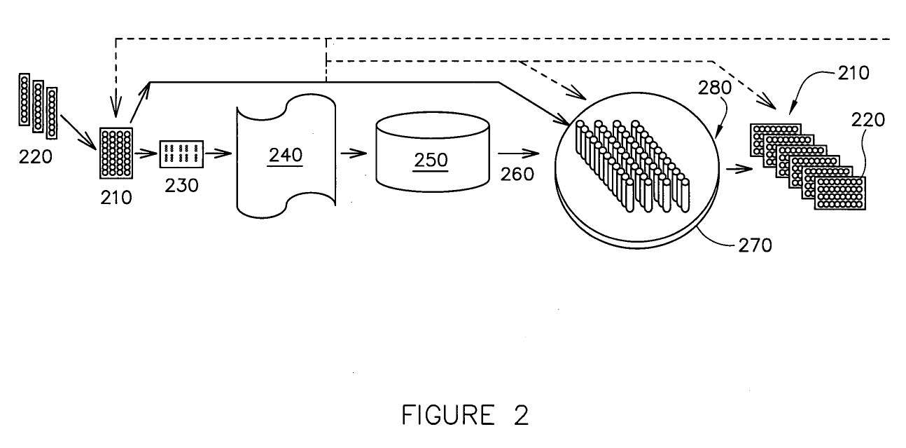

One option for the creation of a library is shown in FIG. 2. Drug samples 220 are formed into a drug sample library 210. The drug sample library 210 is optionally initially tested, for instance in the case of drug candidates, at a polymorph testing station 230 as described in copending U.S. patent application Ser. Nos. 10/156,329, 10/156,245, 10/156,222 and 10/156,295, which are hereby incorporated by reference in their entirety, to determine if the desired drug candidate polymorph members have the desired properties. There can be bulk manufacturing 240 and bulk storage 250 of the drug sample library, so that each member is made in greater quantities and optionally stored for future multiple testing of the same library drug sample member for stability under different exposure conditions of interest or combined with other components for compatibility evaluation. In embodiments using bulk manufacture and storage, the drug sample members in the drug sample library can be in any suitable form. If the drug sample members are in solid form, they can be optionally dissolved or diluted in a suitable solvent in a dissolution or dilution step 260 to provide the drug sample library 210 with member drug samples 280 in a liquid form at point 270 in FIG. 2. Dilution or dissolution can be manual or automatic, such as with known liquid handling robots. Other processing conditions of dissolution or dilution can also be controlled, such as using a glove box for an inert atmosphere during dilution or dissolution. The temperature of the operation can also be controlled by providing a heating or cooling block, such as that disclosed in U.S. Pat. No. 6,528,026, incorporated herein by reference. In other embodiments, a drug sample library is provided in liquid form, for example with each drug sample stored in a separate vial. In those embodiments, the drug sample members can each be stored in a vial having a septum that can be penetrated by a needle mounted on the robotic arm of a liquid handling robot. Optionally, as shown in FIG. 2 also, the bulk manufacturing or storage steps can be eliminated so that the drug sample library 210 goes directly from preparation to point 270 in FIG. 2.

In typical embodiments, drug sample libraries are evaluated for the stability of drug candidates or the compatibility of drug compositions when exposed to various conditions over time. The exposure conditions can be chemical, environmental, or a combination. Examples of chemical exposure conditions that can affect the stability of a drug sample include the presence of acids, bases, oxidants and radical generators. Examples of environmental exposure conditions that can affect the stability of a-drug sample include, heat, light, atmosphere and humidity.

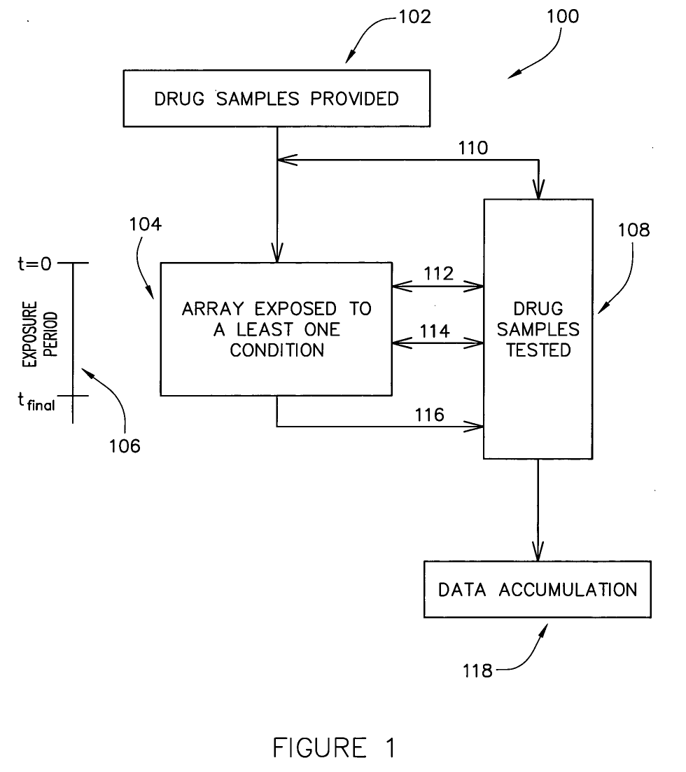

A first workflow according to the invention can be employed: 1) for evaluating the stability of a drug candidate or mixture of drug candidates when exposed to conditions over time; 2) for evaluating the compatibility of drug composition components, specifically drug candidates and excipients, when exposed to conditions over time; and/or 3) for evaluating both the stability of a drug candidate or mixture of drug candidates and the compatibility of drug composition components when exposed to conditions over time, as shown in FIG. 1.

In this first workflow methodology, a drug sample is dispensed or provided, or a plurality of drug samples are dispensed or provided as a library in an array format. The library can differ by drug candidate type, amount, form, or number, as well as by excipient type and amount. Also, in embodiments where the drug candidates are in a solution (the solvent being an excipient), the concentrations can vary. In one embodiment, the library contains a plurality of different types of drug candidates that can be exposed to the same condition. In another embodiment, each member of the library includes the same drug candidate types and forms, with portions of the library being exposed to different conditions. The drug samples are exposed to at least one condition, which can be either a chemical condition or an environmental condition, and are evaluated for any chemical or physical changes over time. Using this workflow, the stability of drug candidates when exposed to various conditions can be evaluated over time, and drug composition libraries for drug composition component compatibility evaluation can be designed based on those results. Alternatively, or in addition, the compatibility of drug composition components when exposed to a condition can be evaluated over time. In still another alternative, the chemical stability of drug candidates and the compatibility of drug composition components can be evaluated simultaneously when exposed to a condition over time.

FIG. 1 shows a flow diagram of the general overall workflow. As shown in FIG. 1, the drug sample evaluation methodology 100 starts with drug candidate(s) and/or drug composition(s), which can be arranged in array format. The array can contain a plurality of different drug candidate types and/or forms, as well as different types and amounts of excipients. The workflow can employ arrays of drug candidates, drug compositions, or a combination of drug candidates and drug compositions. The drug samples of the array can all be the same, all be different, or have some similar samples with overall diversity in the array. The array can contain any desired combination of drug candidate combinations, with or without excipients. Different types, salts or forms of the drug candidate or combination of drug candidates and drug compositions are synthesized or provided on the array 102. The array is then exposed to at least one condition 104 for a period of time defined by an exposure period 106. The condition can be a chemical condition or an environmental condition. For these types of evaluations, it should be noted that in drug compositions, the excipients can create a chemical condition, since they can have acidic or basic properties, for example. The drug samples are tested 108 at various times throughout the workflow using a suitable test in order to determine a property of the drug samples at a particular time. The testing can occur at a variety of times, such as before exposure 110, during exposure 112, 114 and after exposure 116. Testing can occur in situ, or samples can be removed from the exposure condition and tested between sub periods of exposure. More or fewer tests can occur than are represented in FIG. 1. The samples are tested at least twice in order to generate two data points that can be compared, with at least a portion of the exposure period being between the two tests. A graphical representation or other type of data accumulation can then be generated 118 from the data, showing any change over time in the properties of the drug samples. The data from these evaluation studies is analyzed to determine the stability of the drug samples and/or the compatibility of the drug composition components over time when exposed to various conditions. The process shown in FIG. 1 can be carried out in a high throughput mode, and can be performed on smaller samples. For example, the amount of drug candidate in each sample can be less than 40 mg, less than 20 mg, less than 10 mg, less than 1 mg, or less than 0.1 mg. The steps can be carried out in parallel or rapid serial mode, as discussed in more detail below.

The samples are tested for identification and/or characteristics and the data is analyzed to select a suitable drug candidate and excipient combination. The apparatus and methods described herein provide a high throughput capacity providing the ability to perform additional experiments at a reduced cost, meaning that possibly new and different drug candidates and drug compositions can be evaluated and optimized.

The workflow described above can utilize combinatorial or high throughput methods and apparatus. The combinatorial method typically begins with a selection or identification of the variables that are desired to be observed, such as certain drug candidate activities, as well as excipient properties that are desired in a drug product. After identifying the drug candidate or candidates that will be evaluated, a determination is made whether to evaluate chemical stability of the drug candidate, compatibility of drug composition components, or both. Thereafter, exposure and evaluation can be conducted in a high throughput or combinatorial manner. Using the equipment and methods discussed herein, between 1 and 500 library members can be created and evaluated in from about 10 minutes to about 24 hours depending on the test. More typically, the measure of throughput is based on the number of arrays or libraries that can be created and tested in a work day, with a typical throughput being about 1-5 arrays per work day, with 96 members per array or library. This throughput includes the synthesis and testing of the arrays or libraries as discussed herein. While the preparation and testing can be conducted in a high throughput manner, the exposure portions of the workflow are still constrained by the desired exposure time. An advantage of certain aspects of the invention for this part of the workflow is the ability to simultaneously expose libraries of samples to different conditions, which can make it possible to generate data for tens to hundreds to thousands of samples at a time.

The samples can be prepared to provide solid drug candidates in solution form. One typical solvent used in the invention is water. A variety of co-solvents, such as ethanol, propylene glycol, and glycerin, can be included in an evaluation to boost the solubility of components or to improve the stability. Suitable solvents include those solvents that can be used in oral and parenteral (injectable) formulations, such as acetone, ethanol, benzyl bezoate, corn oil, cottonseed oil, ethyl acetate, glycerin, peanut oil, acetonitrile, tetrahydrofuran, dimethylsulfoxide and sesame oil.

The workflows described herein can be used to evaluate a variety of excipients besides solvents, and can be selected based, for example, on the desired properties and/or forms of a final drug product. For example, different classes of excipients add different properties to drug compositions, such as diluents, disintegrants, colorants, glidants, fillers, lubricants and binders, to name a few. Excipients known to impart these features have been extensively studied and are always being developed. Familiarity with these classes of excipients and understanding of the desired property or properties of the drug composition can aid in the design of the library. Also, any understanding of the stability of the drug candidate or candidates will assist the library designer to select the appropriate excipients for evaluation, since certain excipients are known to impart certain chemical features, such as acidity or alkalinity. A number of solvents and other excipients can be found at the US Pharmacopeia National Formulary (2002) which is hereby incorporated by reference.

The samples can be provided or prepared. Drug samples can be prepared for exposure at a sample preparation station. For example, a drug candidate can be dispensed to a selected number of wells of a substrate (typically all of the wells). If the drug candidate or excipient is in a solid form, dispensing can occur with the use of solid handling equipment, such as the Powdemium, available from AutoDose, Geneva, Switzerland, or the device described in U.S. application Ser. No. 10/460,521, hereby incorporated by reference. Specific equipment that can be used in this process, including microtiter plates and liquid/slurry handling robots, are described below. If the drug candidate is in a solution or slurry, the solvent can be driven off across the plate in parallel if desired (such as by blowing nitrogen over the library or with a solvent evaporator, e.g., Genevac HT-8 (Genevac Inc, Valley Cottage, N.Y. 10989)) under reduced pressure or vacuum. If the drug candidate is dispensed in a solvent that is to be evaluated as an excipient, then solvent removal is unnecessary. After the drug candidate or candidates are dispensed, any additional chemical chosen for chemical exposure, such as an acid or base (in any appropriate form), can be dispensed into the wells of the array. The array can then be processed to expose the drug candidates/compositions to the desired chemical and/or environmental conditions. For example, the array can be sealed and heated, exposed to light, etc., to allow the drug candidates, other components, and added chemicals to interact under the desired conditions.

In one embodiment, an agitator, such as a glass or metal ball can be added to each vial to improve mechanical agitation during the preparation step. The balls can be added one at a time or in parallel using a device such as that described in U.S. patent application Ser. No. 10/156,329.

In one embodiment, an array of excipients, such as solvents or solvent mixtures can be added to the vials, as discussed above. The vials are sealed and transported to the exposure stations. Equilibration between a solid and liquid is accelerated by the use of mechanical and/or thermal agitation. In one aspect, the system is equilibrated through the use of a vortexer, sonicator, shaker and/or incubator. If in a solution, the concentration of the drug candidate in the solution can be measured by any of a variety of techniques including liquid chromatography, gas chromatography, thin layer chromatography, IR or Raman spectroscopy or UV-Vis adsorption (as discussed below).

In order to evaluate drug candidate and/or drug composition samples under a variety of exposure conditions, in one embodiment, daughter libraries can be created from arrays of drug candidates or compositions. A daughter library can be created from a parent library at a daughtering station by taking one or more aliquots from one or more members in the parent library, wherein an aliquot is a definite fraction of a whole. This process is referred to as “daughtering.” Typically, daughtering is done for liquid solutions, due to the abilities of the handling equipment. For example, a liquid pipette, operated either manually or automatically (e.g., robotically), can be used to draw an aliquot of a member from the parent library and dispense that aliquot into another container to give a daughter library member. However, in some cases, daughtering of solid samples can be performed with the appropriate solids handling equipment. A limited number of members of the parent library can be daughtered or all the members can be daughtered at least once to create one daughter library. Thus, a daughter library can be smaller than the parent library in terms of either mass, volume or moles and/or in terms of the number of members. Daughtering is performed, for example, to allow for multiple experiments on an identical set of solutions or samples or to avoid having to recreate the parent library. For example, making daughter libraries allows for exposure of identical libraries to different conditions, such as different environments or different chemical exposures. Daughtering can be performed using known equipment, such as hand pipetters, hand-multichannel pipetters, or robots (such as Matrix, CyberLab, Tecan or Hydra robots or Symyx Core Modules).

Automated equipment can be used to increase the speed of the methodology. Liquid or solid handling robots, such as those available from Cavro Scientific Instruments, Inc. (Sunnyvale, Calif.) are available. In addition, microtiter plates and reactors in microtiter plate format can be used to carry out the methods of the invention.

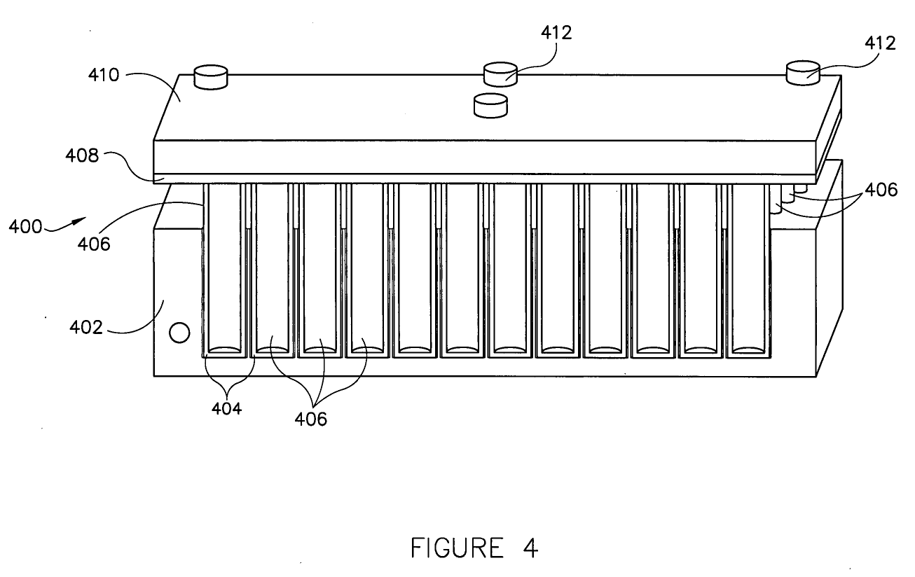

In aspects of the invention that include liquid samples, the regions of the substrate can be wells. The wells can be in a substrate itself (such as in commercially available microtiter plates), but can also be vials or vessels that are placed in a container base (such as that shown in FIG. 4). The use of vials or vessels provides the ability to remove a particular member of the library from the substrate. The vials or vessels can be of a chosen size, such as in the range from about 3 ml to about 200 μl, depending on the desired sample size, with 1 ml or 750 μl vials being commonly used herein. The number of regions (e.g., wells) on the substrate is not critical and can be 8 or more, 16 or more, 20 or more, 24 or more, 32 or more, 48 or more, or 96 or more. The greater the number of regions, the higher throughput that can be achieved in the methodology. The substrate can be any of a wide variety of materials including, for example, polymers, plastics, Pyrex, quartz, resins, silicon, silica or silica-based materials, carbon, metals, inorganic glasses, inorganic crystals, membranes, etc., with metal substrates (particularly aluminum and stainless steel) being used in some aspects of the methodology and optically transparent substrates being used in other aspects of the methodology. The substrate can have any convenient shape, such a disc, square, rectangle, circle, etc. Alternatively, the regions of the substrate can also be planar surfaces with discrete regions enclosed by hydrophobic or hydrophilic borders that prevent the library members from leaving their respective regions.

One example of a sample container in a microtiter plate design is shown in FIG. 4. The sample container 400 has a block 402 with a plurality of wells 404 for receiving a plurality of vessels or vials 406. To optionally seal the vessels 406, a sealing sheet 408 can be placed over the top lip of the plurality of vessels 406 and a cover plate 410 can be fastened to the block 402. Fastening can be by bolts, clips, clamps, wing nuts or other known fastening methods. Bolts 412 are shown in FIG. 4 as the fastening method and the bolts 412 can be screwed into threaded bores drilled into the block 402 to bring the sealing sheet 408 into pressure sealing engagement with the vessels 406. Materials useful as the block and cover plate include aluminum, steel or other metals, with aluminum being preferred for its thermal transfer properties. The vessels 406 can be plastic or glass, with glass being preferred. The sealing sheet 408 can be made from a material that is chemically resistant to the chemicals in the vessels as well as being elastic for its sealing properties. The sheet 408 can be a material such as Teflon®, silicone rubber, Vitron®, Kalrez® or equivalents. Parallel sample containers of this type can be used for the exposure workflow discussed above, and can be heated. Mixing/stirring balls can be added to the parallel sample container by hand or with a device such as that described in U.S. patent application Ser. No. 10/156,329. The container can then be placed on a rocking or rotating or vortexing plate optionally fixed with a heating element for mixing and/or exposing the samples to a controlled temperature. Alternatively, magnetic stirrers (e.g., fleas) can be placed in the vessels and the sample container can be placed on a heater/stirrer plate to afford agitation and/or heating. Also, the heating can be programmable to include desired heating rates or times or temperatures or multi-step profiles, depending on the desired exposure. In other embodiments, the plate can be located in an oven or environmental chamber in which the environmental exposures can occur.

The methodology or workflow can also be performed with a planar substrate for solid samples. Substrates of a variety of materials, such as glass, can be purchased commercially from Zinsser Analytic gmbh (Frankfurt, Germany), which can be, for example, borosilicate reactor plates in the format of the microtiter plates.

Using this equipment, an array of samples can be created, for example, by dispensing the chosen drug candidate and/or excipient according to the library design. In one embodiment, the sample container is sealed so that the individual vessels are each sealed, following which the container is optionally heated and the contents of the vessels are exposed to a controlled temperature for a selected period of time, or an exposure period. The temperature can be typically in the range of from about room temperature (e.g., about 25° C.) to about 10° C. lower than the boiling point of the most volatile chemical in the array and the exposure period can be in the range of from about one hour to about 200 hours or more.

In one embodiment, a solution dispensing station, filtration station and crystallization station, such as those described in U.S. patent application Ser. No. 10/156,329 can be used, alone or in combination, to prepare libraries of drug samples. The drug sample libraries can be placed in an oven or other environmental control apparatus for exposure(such as the Torrey Pines incubator). In some methodologies, the samples are agitated to dissolve as much drug candidate and/or excipient as possible in the solvent(s). In one embodiment a two-arm, three-axis robot having a plurality of pumps and a temperature controlled housing, such as that described in U.S. patent application Ser. No. 10/156,329 can be used.

In one embodiment, the workflow begins with a drug candidate being dispensed into vials, either while the vials are in a separate rack or in a reactor base. The drug candidate can be in a solid state or in solution or suspension, but any solvent present with the original form of the drug candidate can be removed, for example, by evaporation or wicking or other methods known to those of skill in the art. The desired excipients selected as discussed above can be dispensed into each vial in the desired amounts. Optionally, different chemicals for exposure, such as acids, bases, oxidants or radical producing species are added. Also optionally, mixing balls can be placed in the vials (for example, using a device such as that described in U.S. patent application Ser. No. 10/156,329). The assembly can then placed appropriately for the exposure period. If only chemical exposure is desired, the assembly containing the drug samples and the chemicals can be placed on a storage rack or a shelf where it can be easily retrieved or in an approprate chemical exposure area. If an environmental exposure is desired, the assembly can be placed in an environmental chamber, which can expose the assembly to heat, light, atmosphere, and/or humidity, and is optionally placed on a commercially available shaker (available through VWR and made by IKA, MTS, WORKS or Lab-line). Some available environmental chambers are Xenon Test Chambers available from Q-Panel Lab Products in Cleveland, Ohio, and the Pharma Safe System manufactured by Sanyo and available from Integrated Services, TCP Inc. in Palisades Park, N.J. In some embodiments, each sample or groups of samples can be isolated in a separate chamber or separate portion of the chamber and be exposed to different conditions from each other. Such a device is described in U.S. Pat. No. 6,455,316, which is hereby incorporated by reference.

Periodically during the exposure period, the samples are tested. If the samples are in an environmental chamber, they can be removed for testing or can be tested in situ (in the chamber). For destructive testing, such as HPLC or calorimetery (which can be serial, rapid serial or parallel), if it desired to test only a portion of the sample, a needle or pipette can be used to sample the liquids in the vials and aspirating an aliquot of liquid (such as less than 1000 μL or less than 100 μL). The aliquot can be taken by hand or automatically, such as with equipment such as that described in U.S. patent application Ser. No. 10/156,329.

Before, during, and/or after exposure, the drug samples can be tested for properties and characterization. In order to gain enough data to evaluate the stability or compatibility of the components of the drug samples over time, a certain minimum number of at least one type of test should be run, with the number in one aspect being at least two tests, in another aspect at least four tests and in another aspect at least 5 tests. The tests can be run at various times during the exposure period in order to generate data to evaluate any effects the exposure can have on the drug samples over time. The tests can be destructive (where sample is consumed) or can be non-destructive (sample is not consumed). In some embodiments, the tests are not quantitative, but are qualitative. For example, a spectroscopic measurement can be taken with sufficient precision in order to determine if members of the library have changed in the time period since they were initially or last tested. Also for example, the test can determine certain characteristics, without determining every (or even most) characteristic of a sample that one of skill in the art can list (such as chemical structure, morphology, etc.) In this aspect, a high throughput evaluation methodology can be created, giving sufficient information to evaluate the samples over time, while maintaining speed in quality experimentation.

In one embodiment, the evaluation workflow described above can be automated, for example, be performed under control of a programmable computer running an evaluation software program. In particular, software, which is part of the invention described herein, has been developed to automate this workflow by receiving quantitative data from an identity screen (such as Raman, X-ray or IR) and making a relative comparison of data to determine whether any change has occurred in the samples over time.

One embodiment of such software implements a method, which begins by testing for initial information on the drug sample(s) before exposure. This information can be quantitative, qualitative or both. For example, a sample can be run through HPLC (which can be serial, rapid serial, or parallel) in order to obtain the chemical structure of the sample, or can be tested for spectroscopic information. Next, the method loads the data obtained from the identity and/or characterization tests. Preferably, this data includes one or more spectra obtained for each member of the library, for example, raw spectral data (e.g., peak location, height, width or the like) identified by well and column so that each spectrum is identified to a particular member of the library.

The evaluation tests can be HPLC, calorimetry, birefringence, X-ray powder diffraction, FT-Ranan spectroscopy, Raman spectroscopy, UV-Vis absorption, Near IR spectroscopy, IR spectroscopy, dynamic light scattering, fluorescence, or others known to impart the desired information. These tests can be performed in parallel or in a rapid serial mode, such that the testing does not delay the overall workflow. As shown in FIG. 1, one aspect includes running at least one type of test at least 4 different times, including initially, finally and twice during the exposure period. This gives information to evaluate the effects the exposure has on the samples over time. Other tests that can be performed on samples include NMR and elemental analysis. In one aspect, birefringence and Raman spectroscopy can be run in high throughput mode, as described below.

Because drug candidates can be available in very small amounts, in some aspects the samples can be tested using non-destructive tests. The same samples can be tested multiple times throughout the exposure period as opposed to testing a different portion or an aliquot of a sample each time, preserving samples without having to destroy a portion of the sample each time. Also, non-destructive testing can be faster than destructive testing. In one aspect, a destructive and a non-destructive test are run initially, one type of non-destructive test is run one or more times on the sample(s) during the exposure period, and a destructive and non-destructive test are run after the exposure period. This gives the ability to observe both any time dependent changes that can occur throughout exposure, as well as the beginning and ending composition of the sample(s), while conserving sample and having tested the same sample throughout exposure.

In one aspect, solubility tests can be used to initially characterize drug samples before exposure. Solubility can be performed by taking a liquid portion of the sample and subjecting it to a concentration detector to determine the amount of the drug candidate (or salt) in the solvent. The concentration can be detected using liquid chromatography, thin layer chromatography, gas chromatography, absorption in the UV-Vis range, infrared (IR), fluorescence or any other technique that determines concentration known to those of skill in the art. In one embodiment, liquid chromatography with a UV detector at the end can be used to determine the concentration. In one aspect, the measure of the concentration of the drug candidate (or salt thereof) in the solvent can be used as a measure of solubility of the drug candidate (or salt thereof) in the specified solvent at the temperature of sampling and testing. A high throughput solubility test can be performed in a rapid serial mode, with samples being analyzed as fast as one minute per sample. Sampling for the solubility test can be performed at room temperature.

Birefringence of sample(s) can be determined in serial, rapid serial, or parallel for all samples through use of a parallel light rotating and collection device, such as shown in U.S. Pat. No. 6,157,449, which is incorporated herein by reference. In one aspect for operating this equipment, a library of samples is formed in an array on a glass substrate and used with an optical scanner with a transmission attachment to run with 2 crossed polarizers. The scanner offers several advantages over an LED array for this application because individual loading of vials is not required and the entire plate can be imaged directly. With this method, one can distinguish between crystals on the side of the vial, or homogeneously dispersed crystals. Also, one can distinguish between a small amount of very birefringent material, and a large amount of slightly birefringent material. This optical screen can be performed on the entire library at once in either dry or wet mode, depending on the material in the array. Birefringence provides a measure of the amount of anisotropy or orientation within a sample. Crystals are ordered structures, and thus they have a high degree of orientation. Amorphous materials have no longer-range order, and thus are macroscopically isotropic in molecular orientation, resulting in low or zero birefringence values. Based on this theory, birefringence is used as a measure of crystallinity. Also, wet and dry birefringence measurements can be compared to obtain information about possible solvates or hydrates (which can be unstable).

Raman spectroscopy can be performed on a commercially availably unit (e.g., Renishaw, Ramascope), with an X-Y stage that can address the samples in a rapid serial mode. In one aspect, in order to run a high throughput screen, peak assignments can not be performed on the spectra acquired. Instead, the spectra are used as “fingerprints” to compare to spectra obtained from the same sample at different times in order to observe any time dependent changes that have occurred. In other aspects, peak-matching software can be used to make the determination of different identity. The sorting software discussed above can be used also. As above, Raman, IR, X-ray or other fingerprint type spectra can not be quantitatively analyzed, but instead can be used for qualitative determinations about the relative similarities or differences between spectra.

Morphology or crystallinity can also be examined by inspecting each of the regions of the libraries under a microscope, for example, with crossed polarizers. X-ray diffraction can be performed on a Bruker GADDS (Bruker AXS, Madison, Wis.). See also, WO 00/36405, which discloses a method and apparatus of evaluating materials in a high throughput and library format, incorporated herein by reference.

Thus, generally, selected tests, such as those described above, can be used to observe any time dependent changes in the samples. The sample preparation methods can be used to prepare bulk samples of the desired drug samples for additional characterization, such as nuclear magnetic resonance microscopy (NMR) and elemental analysis. One aspect of this workflow is the use of a “universal substrate”, which refers to a substrate having samples thereon in regions and that can be used for a variety of tests (described above) without removal of the samples from the substrate. Thus, a-single substrate (e.g., array of materials) can be tested for birefringence, FT Raman, dynamic light scattering, Raman, X-ray diffraction, UV-Vis, Fluorescence, Near IR and IR, to name a few, without the need to handle each sample.

In addition to predicting room temperature stability, the tests identify any decomposition products and under what conditions these decomposition products emerge. To predict room temperature stability, the change of signal intensity is converted to the degree of decomposition (or percentage of decomposition as a function of decomposition time by calibrating the change with a final destructive TPLC analysis. The rate constant is then calculated from the degree of decomposition versus decomposition time. By comparing the rate of decomposition at different temperatures, the activation energy of the decomposition of a specific drug can be calculated. From the activation energy, the rate of decomposition at room temperature under nominal relative humidity can be derived. From the rate constant at room temperature and nominal relative humidity, the shelf life of the drug can be calculated.

Final measurements can be performed using liquid chromatography. The rapid high performance liquid chromatography (HPLC) methods can be used with an Agilent LC 500 system equipped with PDA (photo-diode array) detectors and ChemStation software (from Agilent Technologies). The efficient separation of the target compound from other interference (such as from solvents and decomposition products) can be achieved, for example, by selecting the proper separation column and optimizing mobile phase composition, gradient profile and column temperature or by choosing an appropriate UV detection wavelength

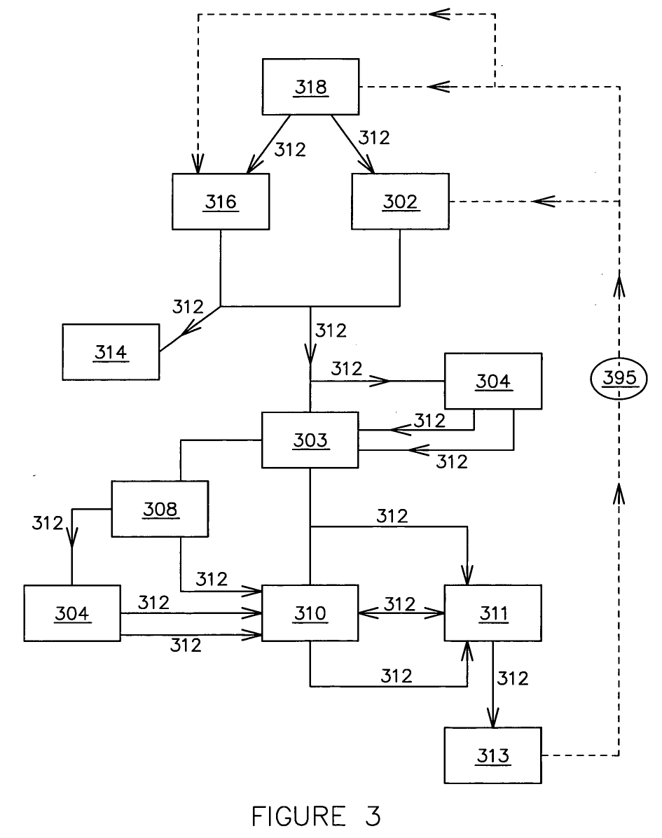

An apparatus or system for evaluating the effects of exposure conditions on drug samples over time is illustrated in FIG. 3. In one aspect, the system 300 includes a parent library 302, a combining station 303, a daughtering station 304 (to create one or more daughter libraries 306), optionally a filtering station 308, an exposure station 310, a testing station 311 and an automated robotic system, represented by arrows 312 to move libraries from one station to another. As used herein a “station” is a location in the apparatus in which one or more functions are performed. The functions can include preparing samples, exposing the sample or samples to an exposure condition, testing the sample(s) or any of the other functions discussed above. Thus, the station can include a liquid handling-robot with pumps and computers to dispense, dissolve, mix and/or move liquids from one container to another. The station can include any of the apparatus discussed above, and can be in an inert atmosphere glove box. Multiple functions can be performed at one location, but for purposes of discussing the methodology in block diagram form, each location or station herein will be referred to as a separate station.

Starting components such as drug candidates and excipients are introduced (or retrieved from storage) and sent to a sample preparation station 303. After preparation, the drug sample library is exposed to an exposure condition for an exposure period at an exposure station 310. The drug sample library can be periodically sent to the testing station 311 for testing as discussed above. A daughtering station 304 can be used to create daughter libraries 306. Finally, a filtering station 308 can be used when solid phase agents are used in the process.

FIG. 3 illustrates a block diagram of an embodiment of a methodology useful in this invention. Starting components 316 or drug candidate and excipient libraries 302 can be maintained in storage 318 and retrieved from storage 318 and moved via the handling system 312 to the sample preparation station 303. In another aspect, samples or libraries are provided. In some aspects, the parent library is daughtered at the daughtering station 304. Multiple paths are shown from the daughtering station 304 to the sample preparation station 303 to show the possibility that multiple daughter libraries are transferred to the sample preparation station 303. The sample preparation station combines the starting components together in a predefined manner, using the components, ratios, etc. as discussed above. Typically, exiting the sample preparation station 303 is either a drug composition library, a drug candidate library, or a library having a combination of drug candidates and drug compositions (all encompassed by the term “drug sample library”). In one aspect, the results of the preparation go to a filtering station 308. Since filtering removes unwanted materials from the library, it can be desirable to daughter the library after filtering, which can be accomplished at a daughtering station 304 between the filtering station 308 and the exposure station 310. The drug sample library proceeds to an exposure station 310, where the library is exposed. In other words, the drug samples of interest are exposed for an exposure period at an exposure station 310, which in some aspects can be a test chamber described above, or can in other aspects can be an area for the samples to sit while they are exposed, for instance to chemicals. Before, during, and/or after exposure, the drug sample library proceeds to the testing station 311, where a test is run to gain initial information, and/or determine the effects of the exposure on the drug samples and/or the qualitative or quantitative degree of change of the samples. The testing station can include a single type of test or multiple types of tests (such as a destructive and a non-destructive test) and can entail using multiple locations for the multiple tests. In one aspect, a feed-back loop 395 is provided that takes testing information 313 from the testing station 311. This testing information can be used at the sample preparation station 303 for new combinations of starting components or creating new drug samples, etc. The feed-back loop 395 can also feed testing information 313 to the starting components or drug sample libraries or the storage location for new drug sample libraries to be created for making a drug sample of interest.

The system 300 includes a computer or processor based system 314 that controls, monitors and/or coordinates the process steps as well as interaction between the various stations 303, 304, 308, 310, 311 and 313. The control system also coordinates the movement of plates (parent or daughter) moving in the robotic system 312. The control system 314 also includes computers, processors and/or software that a user (e.g., chemist) can use to interact with the system 300. Ideally, the control system 314 contains sufficient hardware and software so that it is user-friendly, for example so that the amount of input by the user is limited to the essential design and process elements. The control system 314 can comprise a central computer or processor to command, control and monitor each subsystem or station or piece of the system 300. Alternatively, the control system 314 can comprise an integrated architecture with one or more of the subsystems, stations or pieces that is a smart system of its own right. Thus, a user of the control system 314 can design a set of experiments to create a drug sample library, specify the exposure of that drug sample library and command the system to perform all the chemistry and testing automatically from chemicals in storage.

For example, the control system 314 can command transportation of a library plate from storage to a sample preparation station giving instructions to the sample preparation station that specify the types and volumes of chemicals to dispense. Another example is where similar instructions are used with a daughtering station. The control system 314 can also control the robotics 312 to move chemicals to the various stations 303, 304, 308, 310 and 311. As a further example, the control system 314 can monitor and control the time that a plate remains at a station or the time that an exposure of interest is allowed to occur, such as by instructing a robot to remove the samples at various times. Still further, the control system can monitor and control testing, such as by moving a product library to the testing station and instructing the robot to conduct the test of interest. Additionally, the control system 314 can collect, manipulate and/or store test data. For example, the control system 314 can take data from a test, reduce that data and then send the data for storage to a database. Each sample or each library can be bar-coded, and the control system equipped with an appropriate scanning or reading device for reading the bar code. The control system can then log all of the relevant information for each sample (composition, exposure parameters, time in, time out, test data, test time, etc.). The control system 314 can also monitor the system 300 for safety, problems or other process issues. The control system can also include the feed-back loop 395, discussed elsewhere.

Robotic system 312 can include an automated conveyer, robotic arm or other suitable device that is connected to the control system 314 that can be programmed to deliver the library plate 302 or daughter plates 306 to respective stations 303, 304, 308, 310, 311. The processor can be programmed with the operating parameter using a software interface. Typical operating parameters include the coordinates of each of stations 303, 304, 308, 310, 311 in the system 300 as well as both the library storage plate and daughter plates positioning locations at each station.

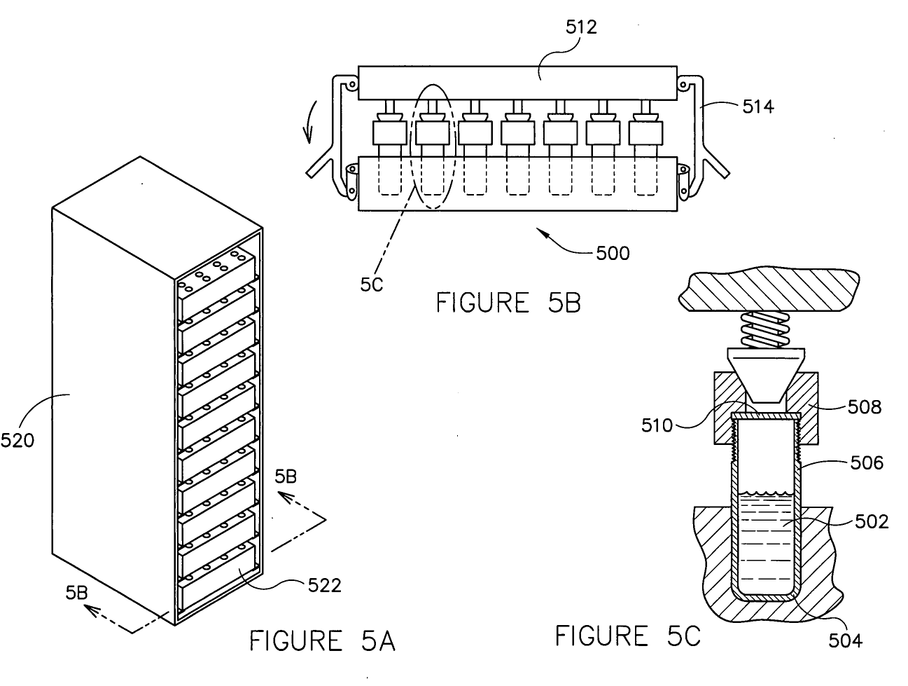

In some embodiments, a library is stored in a storage plate 302, as more clearly seen in FIG. 5B. The library storage plate 500 includes a number of wells 504 formed therein that receive vials 506 containing the library members, as shown in FIG. 5C. Each vial 506 can be provided with a cap 508 having a septum 510 for protecting the members when being stored, (such as a Merlin valve or those described in U.S. application Ser. No. 09/772101 (Patent Application Publication Number 2001/0034067) hereby incorporated by reference. An optional lid 512 having latches 514 shown in FIG. 5B for connecting to the storage plate 502 can also be provided for storage purposes. FIG. 5A also shows that the library plate 522 can be stored in a rack 520 prior to transfer to the next station, such as a sample preparation station or daughtering station.

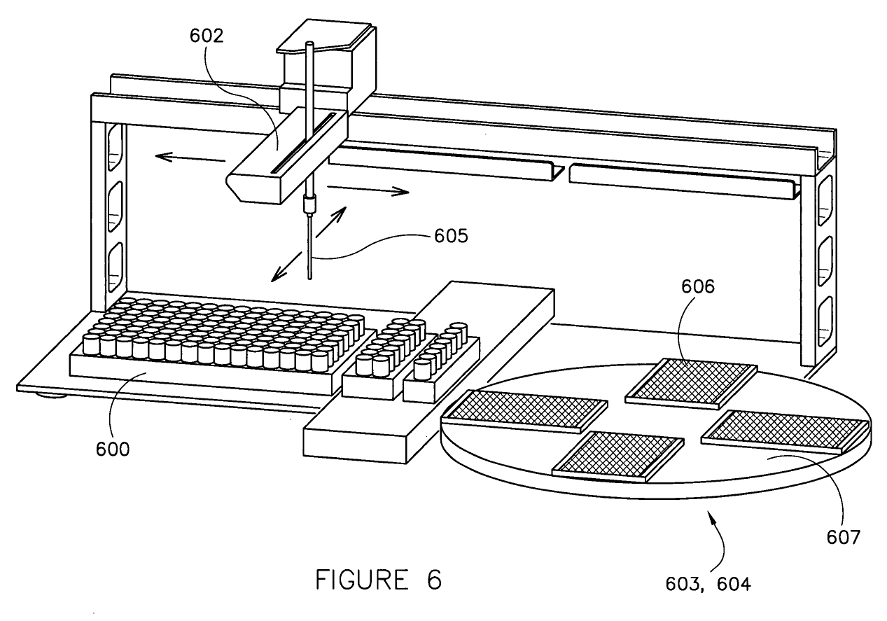

In one embodiment, referring to FIG. 6, a sample preparation station 603 or a daughtering station 604 includes a robotic arm 602 that carries a movable probe 605 and a turntable 607 for holding multiple plates 606 while the preparation or daughtering step is being performed. Robotic arm 602 is movable. The robotic system manipulates the probe 605 using a 3-axis translation system. The probe 605 is movable between vials of drug candidates and excipients 600 arranged adjacent the sample preparation station and plate.

In one aspect, once the drug sample libraries are created, the robotic handling system transports plates to a testing station. As this system can be configured to perform multiple testing steps using one or more types of testing techniques, there can be more than one testing station. Plates containing the libraries can each be receivable in reactor blocks for the testing operation. In one aspect, the plates can be the reactor block that is moved from one station to the next. In one aspect, the reaction block can contain heating elements and temperature sensing devices—thermocouples, thermistors, RTD's and other similar devices—that communication with a processor. The heating elements, temperature sensing devices, and the processor can include a temperature control system that maintains the temperature of each of the drug sample library members at a pre-selected temperature during the exposure period, so that the effects of the temperature on the drug samples over time can be evaluated.

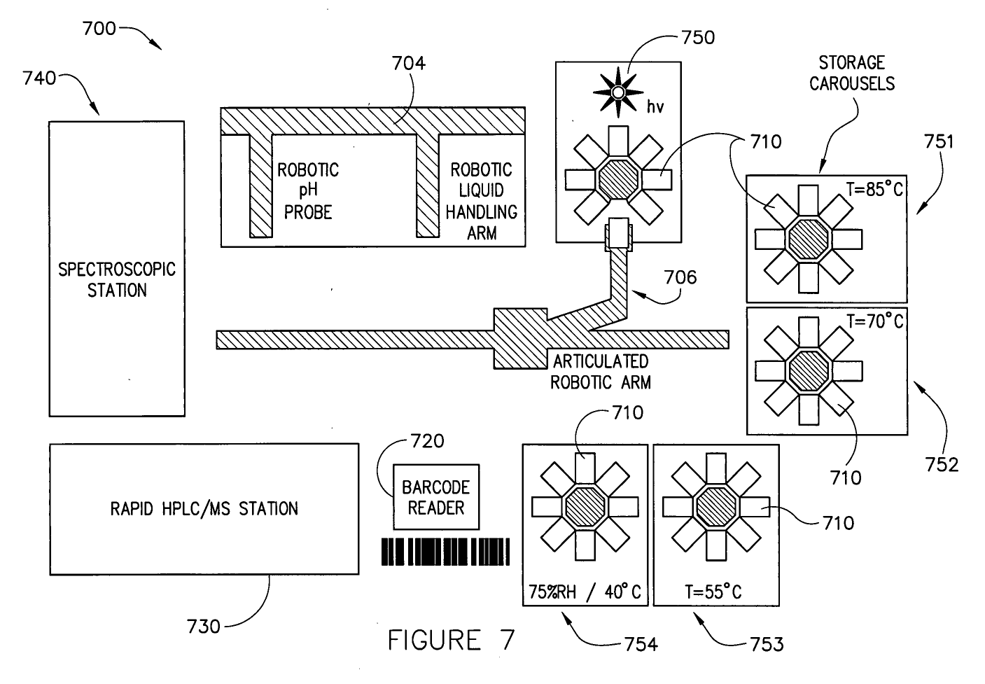

In one embodiment FIG. 7 shows an overall, automated workflow 700 of the invention. Libraries 710 are prepared at the sample preparation station 704 and are optionally daughtered. A robotic arm 706 then transports the libraries 710 between a barcode reader 720 where a bar code is scanned and a log is made, a rapid HPLC station 730 where HPLC analysis are conducted, a spectroscopic station 740, where different spectroscopic tests are run, such as Raman or Near IR, and environmental exposure chambers 750, 751, 752, 753, and 754. In the embodiment shown in FIG. 7, there are 5 exposure chambers 750, 751, 752, 753 and 754, and each contains a carousel 760 that can hold up to 8 libraries of drug samples. Each environmental chamber exposes the libraries to different variables at different limits. As shown in the Figure, one chamber 750 is exposing the libraries to ultraviolet light, one chamber 751 is exposing the libraries to a temperature of 85 C, one chamber 752 is exposing the libraries to a temperature of 70 C, one chamber 753 is exposing the libraries to a temperature of 55 C, and one chamber 754 is exposing the libraries to a humidity of 75% RH and a temperature of 40 C. If each library contains 96 members, it is calculated that 480 samples are being simultaneously exposed. The robotic arm 706 periodically removes libraries for testing at station 740 and replaces them in the chambers, each time running the library by the bar code reader 720 so that a log can be made of the time and circumstances of the transportation.

Software can be used to design, implement and integrate the various pieces of this workflow. As described above, Library Studio™ software can be used to design the libraries of experiments, including, for example drug composition formulas. For a description of the library design software and its capabilities, see, U.S. patent application Ser. No. 09/174,856, filed Oct. 19, 1998 and U.S. patent application Ser. No. 09/420,334, filed Oct. 18, 1999, both of which are incorporated herein by reference for all purposes. This design software outputs a recipe file that can be interpreted by Impressionist® software to create the libraries, as designed. For a description of the library synthesis software and its capabilities, see, U.S. Pat. No. 6,507,945, and WO 00/67086, both of which are incorporated herein by reference. For example, the robots shown in FIG. 5A can be controlled using Impressionist® software. Instruments can be controlled, data acquired, viewed and databased using Epoch™ software from Symyx Technologies, as discussed in U.S. patent application Ser. No. 09/550,549, filed Apr. 14, 2000, which is incorporated herein by reference. The database to store and retrieve data can be based on Oracle® NT database, with other overlays, such as those disclosed in U.S. Pat. No. 6,658,429, which is incorporated herein by reference.

The software aspects herein can be implemented in digital electronic circuitry, or in computer hardware, firmware, software, or in combinations of them. Apparatus of the software inventions can be implemented in a computer program product tangibly embodied in a machine-readable storage device for execution by a programmable processor; and method steps of the invention can be performed by a programmable processor executing a program of instructions to perform functions of the invention by operating on input data and generating output. The invention can be implemented in one or more computer programs that are executable on a programmable system including at least one programmable processor coupled to receive data and instructions from, and to transmit data and instructions to, a data storage system, at least one input device, and at least one output device. Each computer program can be implemented in a high-level procedural or object-oriented programming language, or in assembly or machine language if desired; and in any case, the language can be a compiled or interpreted language. The protocols, procedures, blocks, actions and other objects discussed above can be implemented as component objects implementing an appropriate interface in a component software architecture such as Microsoft Corporation's Component Object Model (COM) or Distributed Component Object Model (DCOM) standards, or the Object Management Group's Common Object Request Broker Architecture (CORBA) standard. Suitable processors include, by way of example, both general and special purpose microprocessors. Generally, a processor will receive instructions and data from a read-only memory and/or a random access memory. Generally, a computer will include one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM disks. Any of the foregoing can be supplemented by, or incorporated in, ASICs (application-specific integrated circuits).

To provide for interaction with a user, the invention can be implemented on a computer system having a display device such as a monitor or LCD screen for displaying information to the user and a keyboard and a pointing device such as a mouse or a trackball by which the user can provide input to the computer system. The computer system can be programmed to provide a graphical user interface through which computer programs interact with users.

Finally, it must be noted that even though many embodiments of the invention are described in a combinatorial manner, that the workflow can be conducted in the same manner for a single sample.

### EXAMPLES

GENERAL: All evaluations were performed with materials that were purchased from Aldrich Chemical in Milwaukee, Wisconsin and used as received.

In the following examples, drug candidate stability is evaluated using non-destructive tests to track any change in property over time while exposed to a condition. Fluorescence, Raman, powder X-ray diffraction, near-IR, mid-IR, UV-Vis absorbance, and colorimetry are used. A change in the peak intensity for these tests is measured for the samples before exposure and during exposure.

### Example 1

### Fluorescence

Two 20 ml vials containing approximately 10 mg each of Amoxicillin, two 20 ml vials containing approximately 10 mg each of Ampicillin trihydrate and two 20 ml vials containing approximately 10 mg each of Caphalexin hydrate were prepared and placed in an oven at 80° C. One vial of each compound was removed from the oven after 15 hours of exposure, and the other vial of each compound was removed from the oven after 41 hours. Approximately 2 mg samples from each of the vials were placed in discreet regions of a Universal ⋄ Substrate™. Approximately 2 mg of unexposed Amoxicillan, Ampicillin trihydrate and Cephalexil hydrate were also placed in discreet regions of the Universal ⋄ Substrate™. The Raman scattering response and the fluorescence emission response of the samples were measured using a Renishaw Raman Spectrometer.

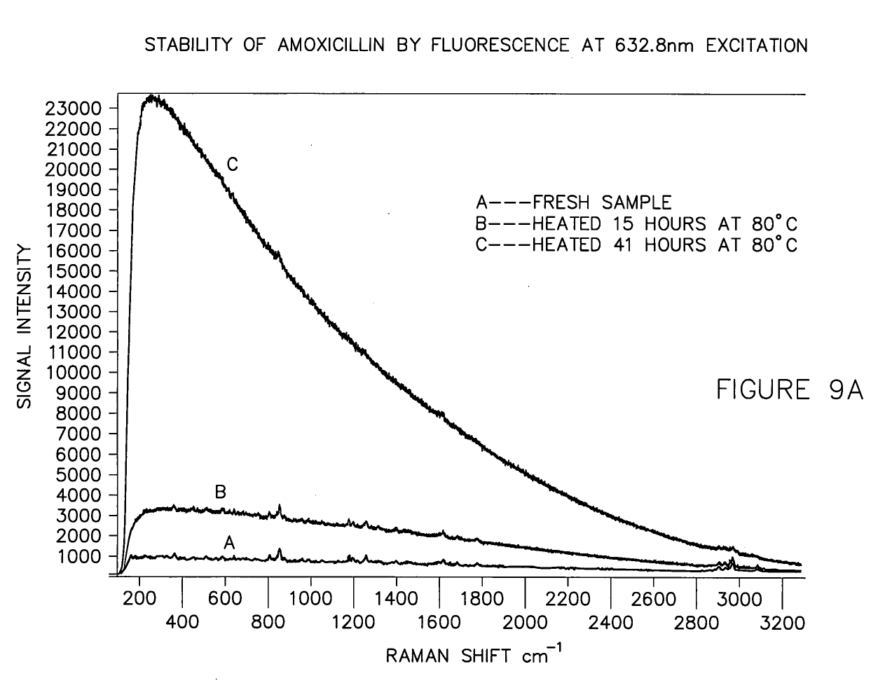

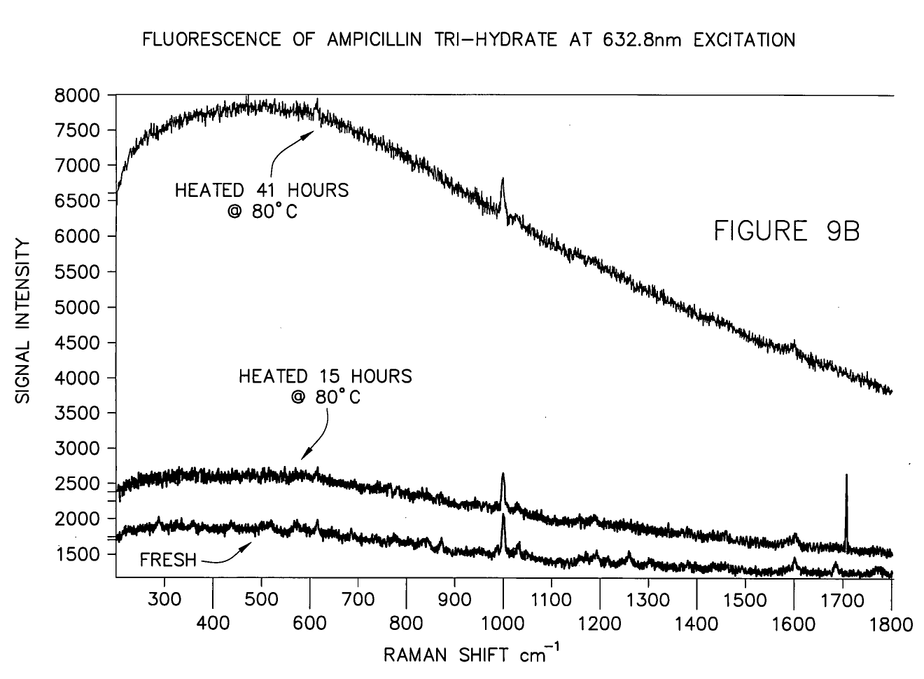

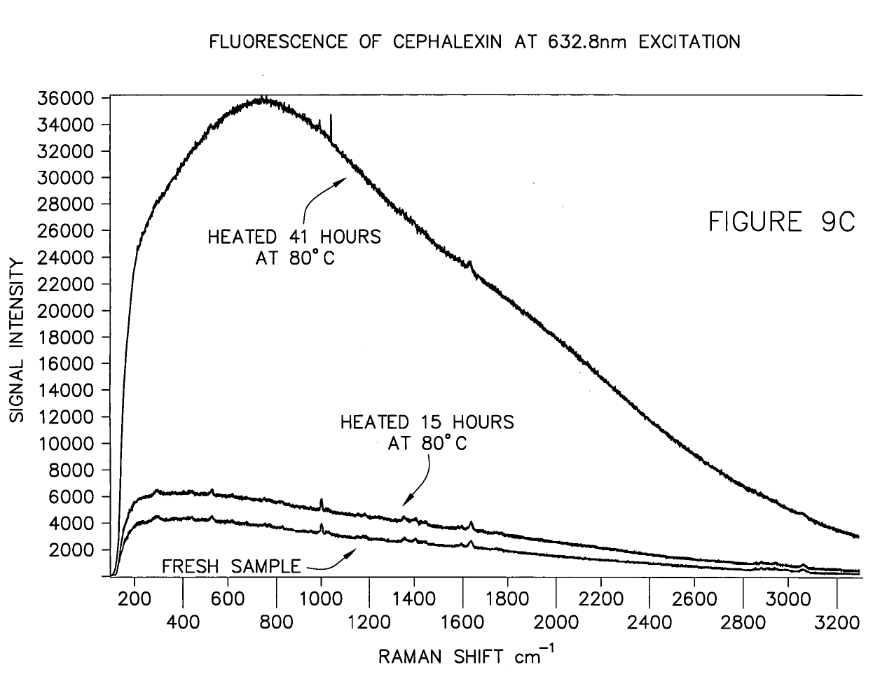

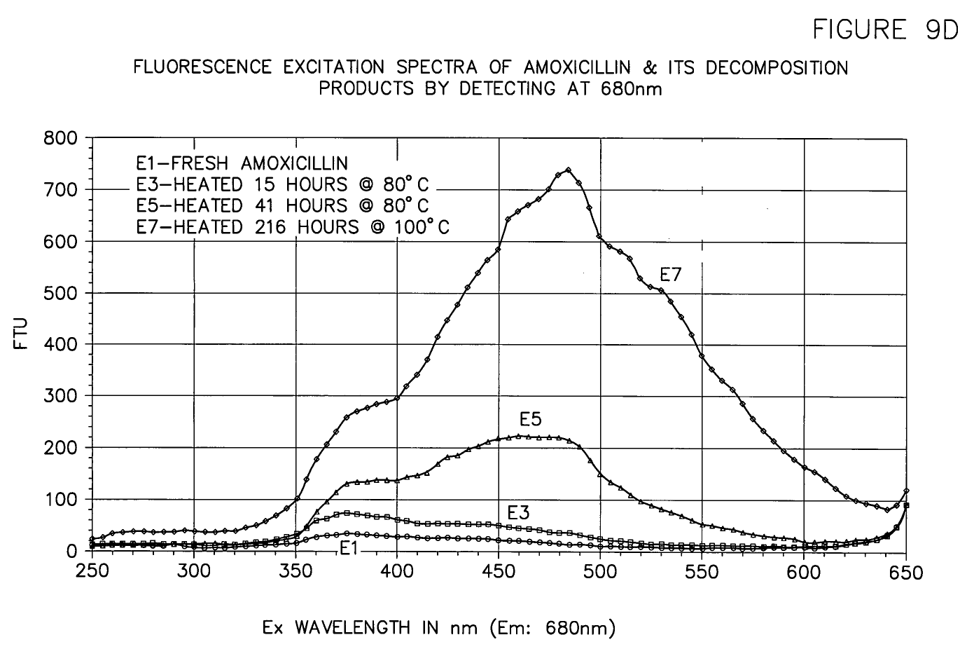

The fluorescence emission of Amoxicillin is shown in FIG. 9a. It can be observed that as the sample was heated, the amplitude of the fluorescence increased significantly as a function of heating time. From the change in fluorescence intensity amplitude as a function of heating time, the time dependent change of Amoxicillin can be seen.

The fluorescence emission of Ampicillin trihydrate is shown in FIG. 9b. It can be observed that as the sample was heated, the amplitude of the fluorescence increased significantly as a function of heating time. From the change in fluorescence intensity amplitude as a function of heating time, the time dependent change of Ampicillin trihydrate can be seen.

The fluorescence emission of Cephalexin hydrate is shown in FIG. 9c. It can be observed that as the sample was heated, the amplitude of the fluorescence increased significantly as a function of heating time. From the change in fluorescence intensity amplitude as a function of heating time, the time dependent change of Cephalexin hydrate can be seen.

FIG. 9d shows the fluorescence excitation spectra for Amoxicillin samples aged up to 216 hours. Fresh samples as well as samples exposed for 80 C at 15, 41 and 216 hrs were compared. The samples were exposed to excitation wavelengths ranging from 250 to 650 nm with the fluorescence response at 680 nm being monitored. A SpectraMax Gemini EM (Molecular Devices) with two scanning monochromators was used.

### Example 2

### FT Raman

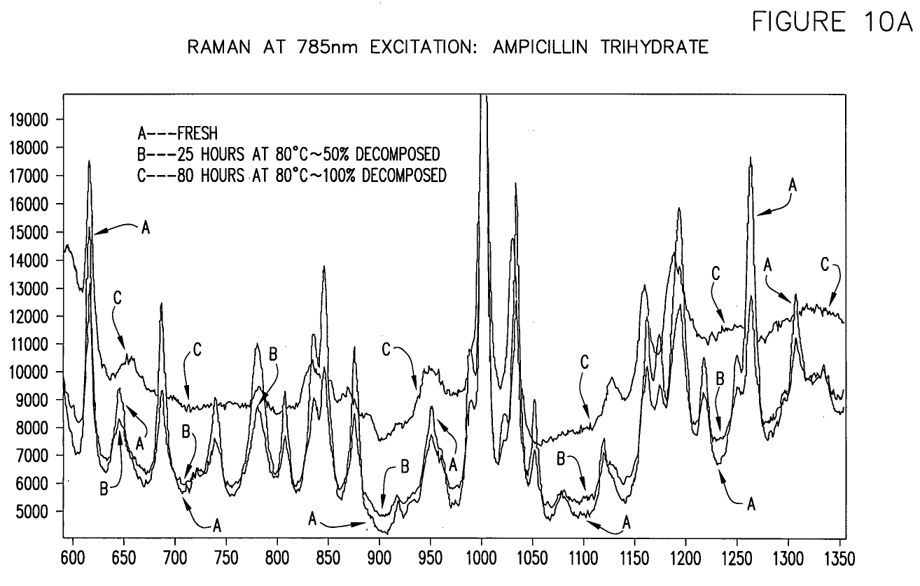

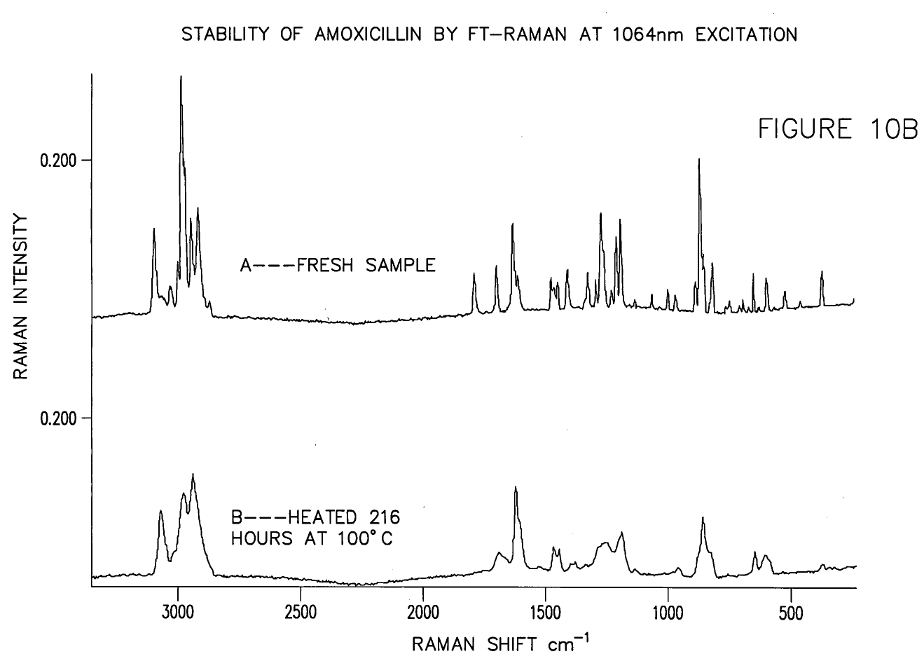

Approximately 2 mg of Ampicillin trihydrate was placed in a 20 ml vial and heated in a lab oven set to 80° C. for approximately 80 hours. FT-Raman responses were measured on an Equinox 55 (Bruker Optics) coupled to Raman module FRA 106/S for 1 mg samples of unexposed Ampicillin trihydrate and 1 mg samples exposed to 80° C. for 25 hours and 80 hours. FIG. 10a shows the FT-Raman spectrum for the three.

FIG. 10b shows the FT-Raman spectra for two samples of Amoxicillin described in Example 1. It can be seen that the spectrum changes significantly for Amoxicillin after being heated for 216 hours at 80° C. The change in FT-Raman signal is used to measure the rate of drug decomposition and the chemical stability of the drug candidate.

FIG. 10c shows the FT-Raman data for Cephalexin hydrate which was exposed to 80 C in a lab oven. Testing was conducted on 1 mg samples of unexposed drug, and samples exposed for 25 and 80 hours.

### Example 3

### Powder X-ray Diffraction

Approximately 2 mg of Amoxicillin was placed on a Universal ⋄ Substrate™ and exposed in an oven for 216 hours at 100 C. After the sample was removed from the oven, approximately 2 mg of unexposed sample was also on the placed on the Universal ⋄ Substrate™. The X-ray powder diffraction (XRD) patterns for the exposed and unexposed samples were collected on a Bruker D-8 C2 X-ray diffractometer.

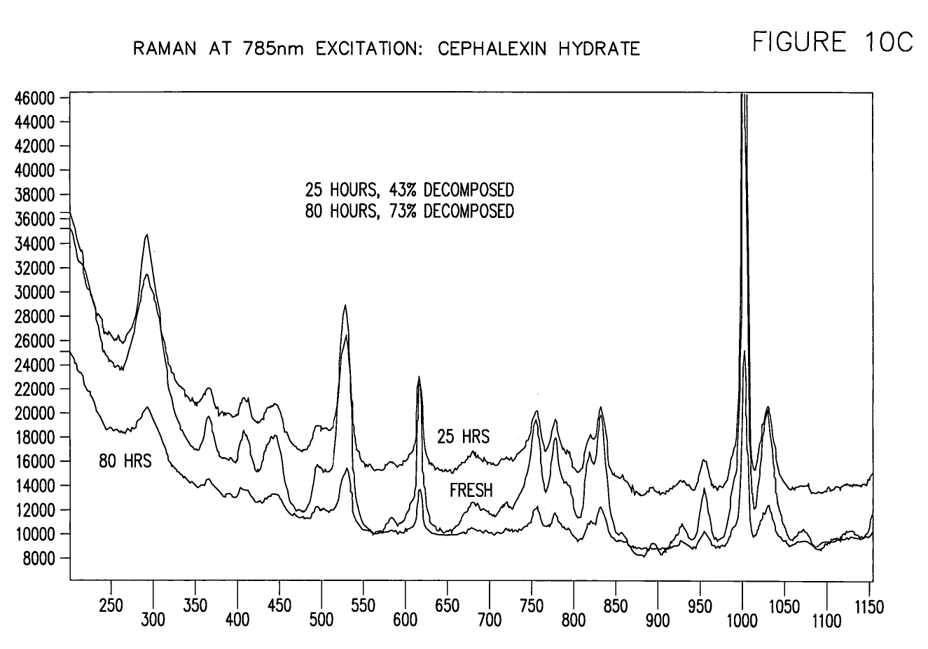

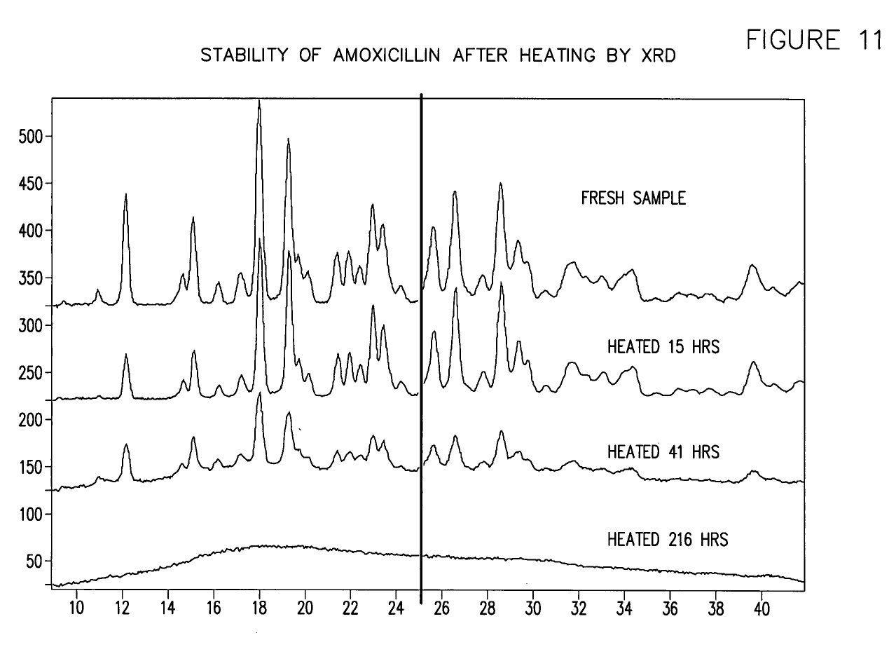

FIG. 11 shows the comparison of the powder diffraction intensity of exposed and unexposed Amoxicillin. It can be observed that the crystallinity is almost completely lost over 216 hours. The rate of the crystallinity loss can be calculated and a value can be derived for room temperature. This in turn can be used to determine the chemical stability and to calculate the shelf lifetime of the drug at room temperature.

### Example 4

### FTNIR

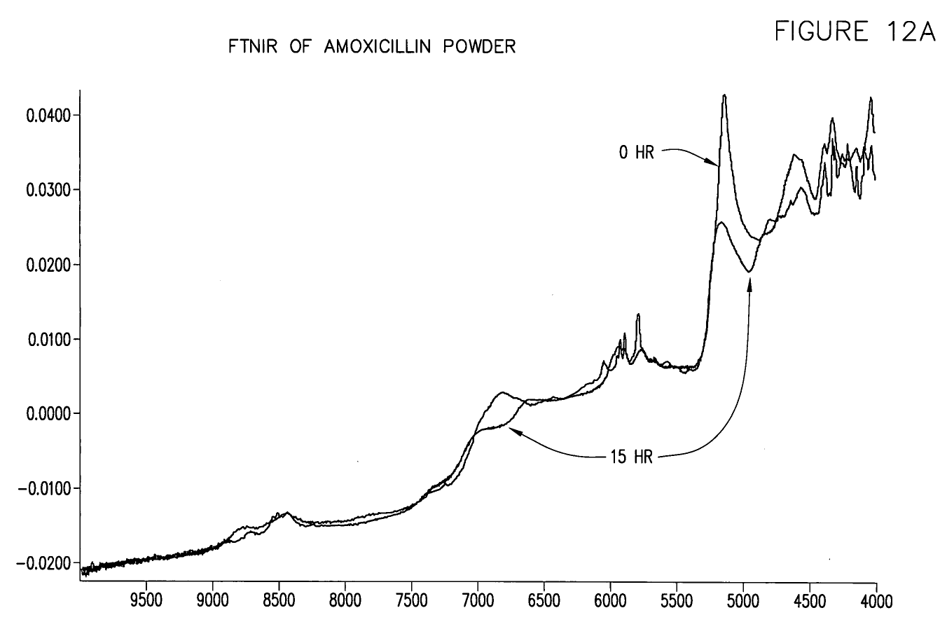

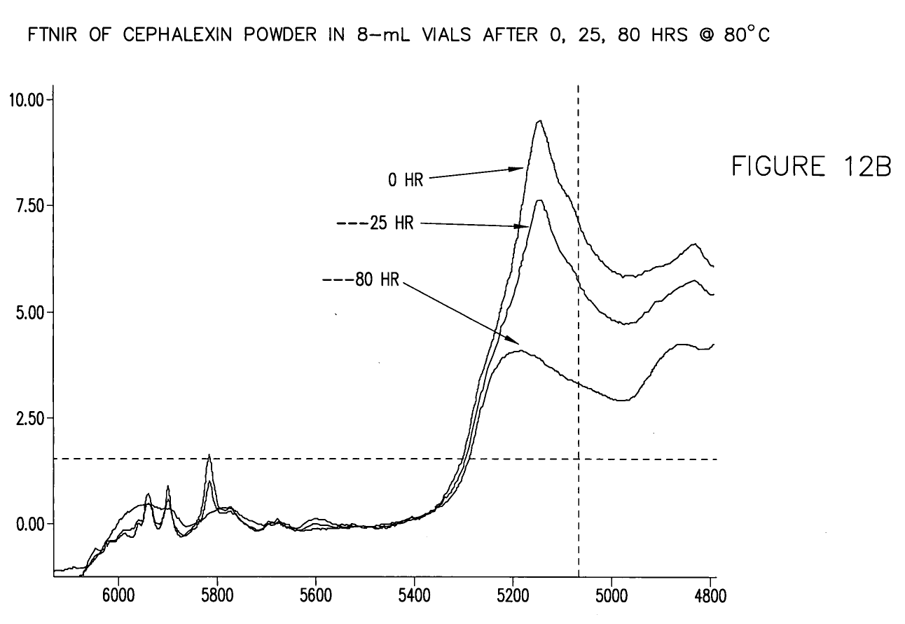

Approximately 2.5 g of Amoxicillin was weighed into an 8-mL vial. 3 mL of an HCl solution of pH 1.0 was added to the vial. The vial was capped and heated to 80 C for 15 hours. The vial was then removed from the oven and water in the vials was removed by blowing a small stream of nitrogen into the vial. After the powder dried up, it was crushed in a mortar and the decamped powder was used for further nondestructive testing. The FTNIR analysis was run on a Bruker FT-NIR spectrometer MPA with a fiber optics module. The results are shown in FIG. 12A.

Approximately 2.5 grams of Cephalexin was weighed into 3 8-mL vials. Two of the samples were capped and heated to 80 C. One sample was removed after 25 hours and the other was removed after 80 hours. The third sample was not heated up. FTNIR analysis was run on the three samples using the device described above. The results are shown in FIG. 12B.

### Example 5

### FTMIR

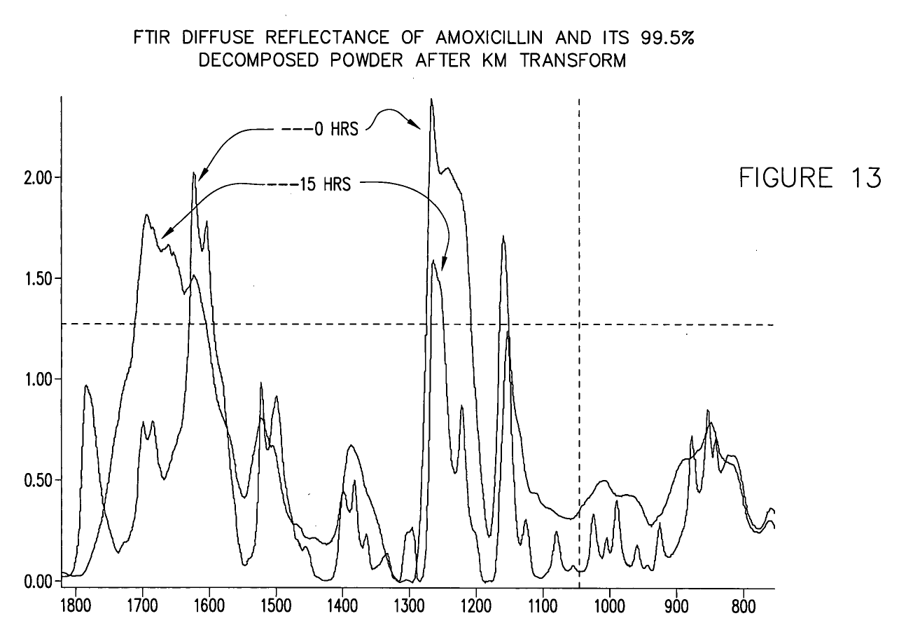

Approximately 2.5 g of Amoxicillin was weighed into an 8-mL vial. 3 mL of an HCl solution of pH 1.0 was added to the vial. The vial was capped and heated to 80 C for 15 hours. The vial was then removed from the oven and water in the vials was removed by blowing a small stream of nitrogen into the vial. After the powder dried up, it was crushed in a mortar and the decamped powder was used for further nondestructive testing. FTMIR analysis was run on a Bruker Equinox 55 FTIR system coupled with an external integration sphere module. The results are shown in FIG. 13.

### Example 6

### UV-Vis Diffuse Reflectance

Approximately 2.5 g of Amoxicillin was weighed into 6 8-mL vials. The vials were capped and heated to 80 C. Vials were removed at 26, 48, 74, 158, and 475 hours. UV-Vis diffuse reflectance was run on the samples using a Cary 50 (Varian) scan spectrophotometer system with a remote DRA. The results are shown in FIG. 14.

### Example 7

### Colorimetry

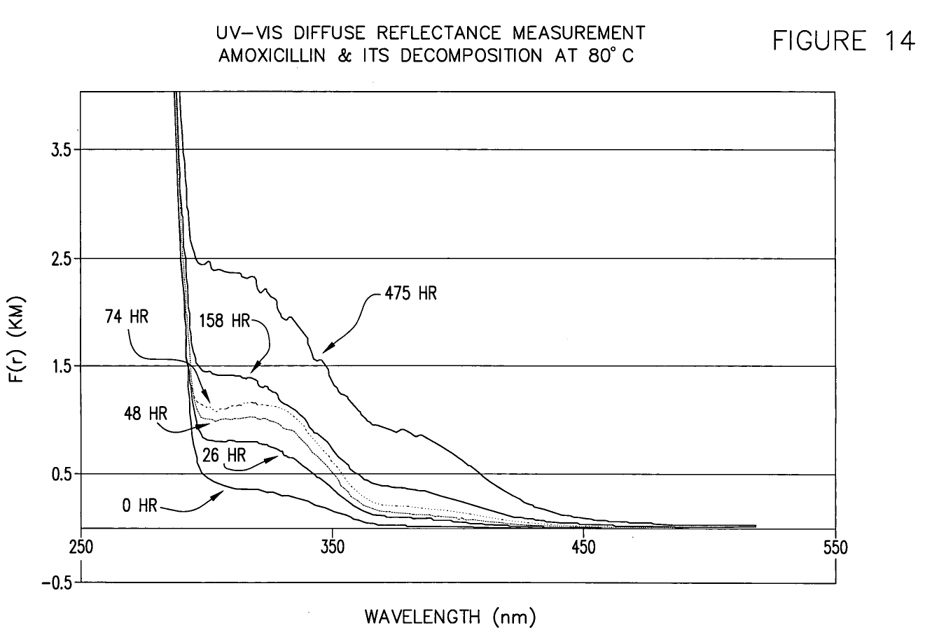

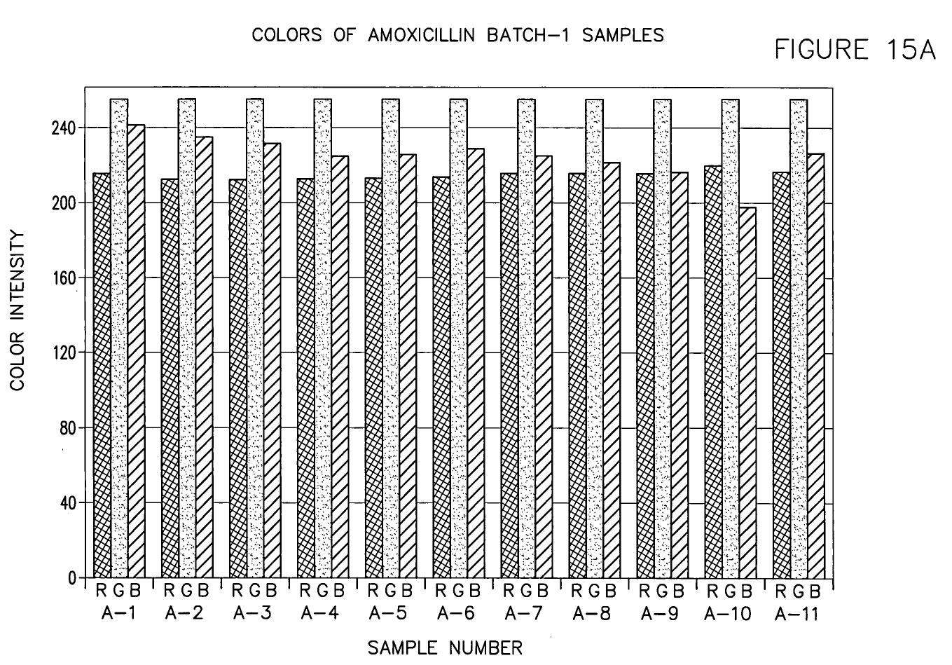

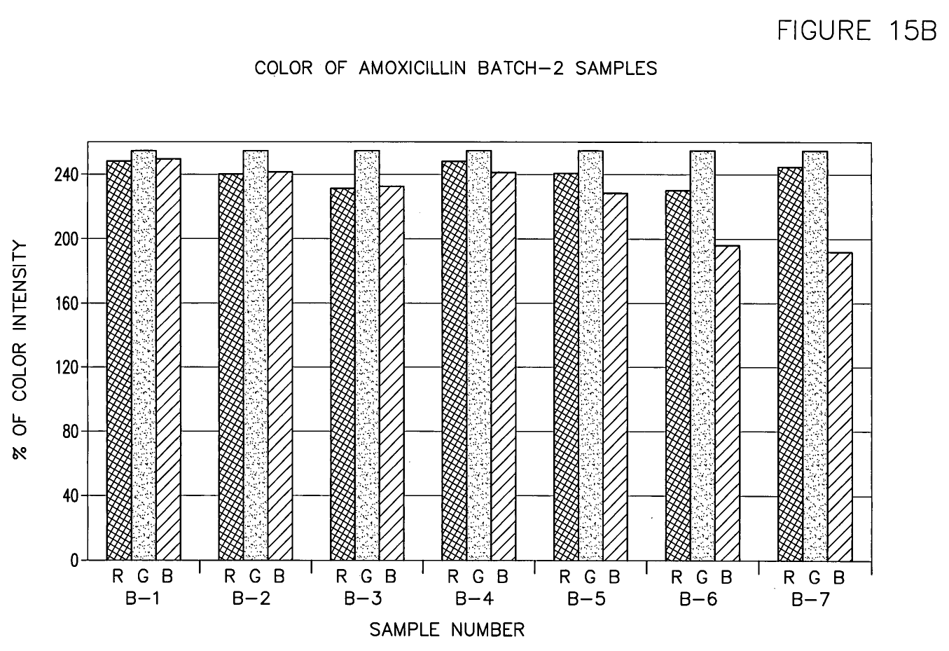

Eleven 20 ml glass vials containing approximately 10 mg each of Amoxicillin were prepared and placed in an oven at 80° C. The samples were removed from exposure at various times as shown in Table 1. After exposure, all of the samples were placed on a quartz substrate and a digital image of the bottom of the vials was taken from beneath the substrate using a digital camera available from Canon. Colorimetry was performed using image analysis software (PMAQ) available from National Instruments. RGB values were extracted from a pre-defined area of each sample image. The results are shown in FIG. 15A.

TABLE 1

| Sample Number | Hours exposed at 80° C. |
|---|---|
| A-1 | 0 |
| A-2 | 14 |
| A-3 | 26 |
| A-4 | 38 |
| A-5 | 48 |
| A-6 | 62 |
| A-7 | 74 |
| A-8 | 110 |
| A-9 | 158 |
| A-10 | 475 |
| A-1l | 62 |

Seven 20 ml glass vials containing approximately 10 mg each of Amoxicillin decomposed to a known amount (i.e., having a certain percentage of the product hydrolyzed) were prepared as shown in Table 2. All of the samples were placed on a quartz substrate and a digital image of the bottom of the vials was taken from beneath the substrate using a digital camera available from Canon. Colorimetry was performed on the samples using the software described above. RGB values were extracted from a pre-defined area of each sample image. The results are shown in FIG. 15B

TABLE 2

| Sample | % hydrolyzed |
|---|---|
| B-1 | 0 |
| B-2 | 1.9 |
| B-3 | 3.0 |
| B-4 | 4.5 |
| B-5 | 9.6 |
| B-6 | 17.3 |
| B-7 | 31.7 |

The accompanying Figures and this description depict and describe embodiments of the system and method of the present invention, and features and components thereof. Fastening, mounting, attaching or connecting the components of the present invention to form the apparatus or device as a whole, unless specifically described otherwise, are intended to encompass conventional fasteners such as machine screws, nut and bolt connectors, machine threaded connectors, snap rings, clamps such as screw clamps and the like, rivets, nuts and bolts, toggles, pins and the like. Components can also be connected by welding, friction fitting or deformation, if appropriate. Electrical connections, if any, can be made using appropriate electrical components and connection methods, including conventional components and connectors. Unless specifically otherwise disclosed or taught, materials for making components of the present invention are selected from appropriate materials such as metal, metallic alloys, fibers, plastics and the like, and appropriate manufacturing or production methods including casting, extruding, molding and machining can be used.

Any references herein to front and back, right and left, top and bottom, upper and lower and horizontal and vertical are intended for convenience of description only, not to limit the present invention or its components to any one positional or spatial orientation. Such terms are to be read and understood with their conventional meanings. In the Figures, elements common to the embodiments of the invention are commonly identified.

A number of embodiments of the invention have been described. Nevertheless, it will be understood that various modifications may be made without departing from the spirit and scope of the invention. For example, in some implementations method steps can be performed in a different order than that disclosed. Similarly, while the workflows and techniques have been described as involving libraries or arrays containing a plurality of samples, the workflows and techniques can be advantageously applied to the evaluation of single samples. Accordingly, other embodiments are within the scope of the following claims.

1-27. cancelled.

28. A method for evaluating the stability of drug samples when exposed to various controlled conditions, the method comprising:
providing an array of drug samples;
simultaneously exposing a plurality of the drug samples to at least one controlled environmental condition for an exposure period;
simultaneously exposing the plurality of the drug samples to at least one controlled chemical condition for the exposure period; and
evaluating any change of the exposed drug samples.

29. The method of claim 28, wherein the plurality of drug samples exposed to the controlled environmental condition and the plurality of drug compositions exposed to the chemical condition are drug candidates.

30. The method of claim 28, wherein the plurality of drug samples are exposed in a chamber.

31. The method of claim 28, wherein the controlled environmental exposure and the controlled chemical exposure occur simultaneously.

32. The method of claim 28, wherein at least two of the drug samples of the array are different from each other.

33. The method of claim 28, wherein at least one of the drug samples is exposed to a first controlled chemical condition and at least one other drug sample is exposed to a second controlled chemical condition different from the first controlled chemical condition.

34. The method of claim 28, wherein at least one of the drug samples is exposed to a first controlled environmental condition and at least one other drug sample is exposed to a second controlled environmental condition different from the first controlled environmental condition.

35. The method of claim 28, wherein the drug samples are drug compositions.

36. The method of claim 28, wherein at least one of the drug samples is a drug candidate and at least one of the drug samples is a drug composition.

37. The method of claim 28, wherein the change in the exposed drug samples is a change in chemical composition of an active pharmaceutical ingredient.

38. The method of claim 28, wherein the change in the exposed drug samples is a change in biological activity of the drug candidate.

39. The method of claim 28, wherein the change in the exposed drug samples is a change in component compatibility.

40. The method of claim 28, wherein a plurality of the drug samples of the array comprise a chemical selected from the group consisting of acids, bases, radicals and oxidizers.

41. The method of claim 28, wherein the controlled environmental condition is selected from the group consisting of heat, humidity and light.

42. The method of claim 28, wherein the drug samples of the array all have chemical or physical diversity.

43. The method of claim 28, wherein the drug compositions of the array are the same.

44. The method of claim 42, wherein at least one of the drug samples of the plurality of drug samples exposed to at least one controlled chemical condition is exposed to a first controlled chemical condition and at least one other drug sample of the plurality of drug samples exposed to at least one controlled chemical condition is exposed to a second controlled chemical condition different from the first controlled chemical condition.

45. The method of claim 43, wherein at least one of the drug samples of the plurality of drug samples exposed to at least one environmental condition is exposed to a first controlled environmental condition and at least one other drug sample of the plurality of drug samples exposed to at least one controlled environmental condition is exposed to a second controlled environmental condition different from the first controlled environmental condition.

46. The method of claim 42, wherein at least one of the drug samples of the plurality of drug samples exposed to at least one controlled environmental condition is exposed to a first controlled environmental condition and at least one other drug sample of the plurality of drug samples exposed to at least one controlled environmental condition is exposed to a second controlled environmental condition different from the first controlled environmental condition.

47. The method of claim 45, wherein at least one of the drug samples of the plurality of drug samples exposed to at least one controlled chemical condition is exposed to a first controlled chemical condition and at least one other drug sample of the plurality of drug samples exposed to at least one controlled chemical condition is exposed to a second controlled chemical condition different from the first controlled chemical condition.

48. The method of claim 28, further comprising testing the exposed drug samples at least twice, wherein at least one of said tests is performed during the exposure period.

49. The method of claim 48, wherein the testing is non-destructive.

50. The method of claim 49, wherein the non-destructive test is selected from the group consisting of raman spectroscopy, X-ray diffraction, near infrared spectroscopy, dynamic light scattering and ultraviolet-visible spectroscopy.

51. The method of claim 48, further comprising conducting a destructive test after the exposure period.

52. The method of claim 48, wherein the testing is destructive.

53. The method of claim 28, further comprising preparing the array of drug samples.

54. The method of claim 28, further comprising daughtering the array into at least four additional arrays before exposure, resulting in at least a first array, a second array, a third array, a fourth array and a fifth array.

55. The method of claim 54, wherein a plurality of the drug samples of the first array is exposed to a first controlled temperature condition, a plurality of the drug samples of the second array is exposed to a second controlled temperature condition different from the first temperature condition, a plurality of the drug samples of the third array is exposed to a first controlled humidity condition, a plurality of the drug samples of the fourth array is exposed to a second controlled humidity condition different from the first humidity condition and a plurality of the drug samples of the fifth array is exposed to a controlled light condition.

56. The method of claim 28, wherein the array is located on a common substrate.

57. The method of claim 56, wherein each drug sample of the array is located on a spatially discrete region of the substrate.

58. The method of claim 28, wherein the drug compositions of the array contain no more than 10 mg of active pharmaceutical ingredient.

59. The method of claim 28, wherein a plurality of the drug samples of the array comprise an excipient selected from the group consisting of lubricants, surfactants, diluents, binders, fillers and disintegrants.

60. The method of claim 28, wherein a plurality of the drug samples of the array comprise a chemical selected from the group consisting of acids, bases, radicals and oxidizers.

61. The method of claim 28, further comprising placing the array of drug samples in a an exposure test chamber.

62-88. cancelled.

89. A method for evaluating the possible effects of a controlled exposure condition on a drug sample, the method comprising:
providing an array of drug samples on a single substrate;
exposing the array and the substrate to at least one controlled condition for an exposure period; and
evaluating the array of drug samples at least twice using one type of test with at least a portion of the exposure period being between the two tests to determine the effects of the exposure on the drug samples of the array, wherein the drug samples of the array remain on the substrate throughout the evaluation step.

90. The method of claim 89, further comprising placing the array of drug samples in an environmental chamber prior to the exposing step.

91. The method of claim 90, wherein the tests are conducted inside the environmental chamber.

92. The method of claim 90, wherein the array of drug samples is removed from the environmental chamber for testing the drug compositions, and replaced in the environmental chamber after the tests.

93. The method of claim 89, wherein the one type of test is a non-destructive test.

94. The method of claim 93, wherein the non-destructive test is selected from the group consisting of raman spectroscopy, X-ray diffraction, near infrared spectroscopy, dynamic light scattering and ultraviolet-visible spectroscopy.

95. The method of claim 93, further comprising evaluating the composition of the array of drug samples after the exposure period using at least one destructive test.

96. The method of claim 95, wherein the destructive test is liquid chromatography.

97. The method of claim 95, wherein the at least one destructive test is conducted in parallel.

98. The method of claim 89, further comprising simultaneously exposing the array of drug samples to at least one controlled chemical condition.

99. The method of claim 89, further comprising daughtering the array into at least four additional arrays before exposure, resulting in at least a first array, a second array, a third array, a fourth array and a fifth array.

100. The method of claim 98, wherein a plurality of the drug samples of the first array is exposed to a first controlled temperature condition, a plurality of the drug samples of the second array is exposed to a second controlled temperature condition different from the first controlled temperature condition, a plurality of the drug samples of the third array is exposed to a first controlled humidity condition, a plurality of the drug samples of the fourth array is exposed to a second controlled humidity condition different from the first controlled humidity condition and a plurality of the drug samples of the fifth array is exposed to a controlled light condition.

101. The method of claim 100, wherein the at least one test is conducted in parallel.

102. The method of claim 89, wherein the at least one test is conducted in parallel.

103. A method of research for possible effects of exposure conditions on a drug sample or a component thereof, the method comprising:
providing an array of drug samples;
simultaneously exposing two samples of the array of drug samples to a set of controlled exposure conditions for a period of time, wherein the controlled exposure conditions vary across the array;
testing the exposed samples; and
determining if there has been any change in the exposed drug samples.

104. The method of claim 103, further comprising placing the array of drug samples in an enclosure prior to exposure.

105. The method of claim 103, wherein the controlled conditions are environmental conditions selected from the group consisting of light, temperature and humidity.

106. The method of claim 103, wherein the array of drug samples are located on a common substrate.

107. The method of claim 103, wherein the method is software integrated.

108. The method of claim 103, wherein the testing is non-destructive.

109. The method of claim 103, wherein the testing is destructive.

110. The method of claim 103, wherein the testing comprises a non-destructive test conducted at least twice on the exposed samples during the period of time, and a destructive test conducted on the exposed samples after the period of time.

111. The method of claim 110, wherein the exposed samples are tested in parallel.

112-134 are cancelled.
